(19) United States
(12) Patent Application Publication
(10) Pub. No.: US 2011/0152147 A1
(43) Pub. Date: Jun. 23, 2011

(54) ENCAPSULATES

(76) Inventors: Johan Smets, Lubbeek (BE); An Pintens, Brasschaat (BE); Olav Pieter Dora Tony Keijzer, Itterbeek (BE); Jean-Francois Bodet, Waterloo (BE); Ariel Lebron, Cincinnati, OH (US); Emiliano Fratini, Sesto (IT); Chiara Vannucci, Pistoia (IT); Moira Ambrosi, Sesto (IT); Piero Baglioni, Fiesole (IT); Sandra Jacqueline Guinebretiere, Appleton, WI (US); Nianxi Yan, Appleton, WI (US); Hongwei Liu, Appleton, WI (US)

(21) Appl. No.: 12/969,817

(22) Filed: Dec. 16, 2010

Related U.S. Application Data
(60) Provisional application No. 61/287,864, filed on Dec. 18, 2009.
(60) Provisional application No. 61/321,986, filed on Apr. 8, 2010.

(51) Int. Cl. C11D 17/00; D06L 1/16; A61K 8/11; C11D 3/60; A61Q 5/02; A61K 8/84; B01J 13/04; B08B 3/00

(52) U.S. Cl. 510/119; 8/137; 510/320; 510/321; 510/403; 510/418; 510/439; 510/516; 264/4.3; 134/26; 132/200

(57) ABSTRACT

Encapsulates, compositions, packaged products and displays comprising such encapsulates, and processes for making and using such encapsulates, compositions, packaged products and displays. Such compositions have improved deposition and retention properties that may impart improved benefit characteristics to a composition and/or situs.

### CROSS-REFERENCE TO RELATED APPLICATIONS

This application claims priority under 35 U.S.C. §119(e) to U.S. Provisional Application No. 61/287,864, filed Dec. 18, 2009, and to U.S. Provisional Application No. 61/321,986, filed Apr. 8, 2010.

### FIELD OF INVENTION

The present application relates to encapsulates, compositions, products comprising such encapsulates, and processes for making and using such encapsulates.

### BACKGROUND OF THE INVENTION

Benefit agents, such as a perfumes, dyes, optical brighteners, fabric care agents, bleaching agents, metal catalysts, bleach boosters, solvents, enzymes, insect repellants, silicones, waxes, flavors, vitamins, cooling agents, and skin care agents are expensive and may be less effective when employed at high levels in compositions such as personal care compositions, cleaning compositions, and fabric care compositions. As a result, there is a desire to maximize the effectiveness of such benefit agents. One manner of achieving such objective is to improve the delivery efficiencies of such benefit agents. Unfortunately, it is difficult to improve the delivery efficiencies of benefit agents as such agents may be lost do to the agents' physical or chemical characteristics, such agents may be incompatible with other compositional components or the situs that is treated, or such agents may be lost during post application processes such as rinsing or drying.

One method of improving the delivery efficiency of a benefit agent is to encapsulate so that the agent is only released, for example by fracturing the shell of the encapsulate, when the benefit agent is desired. However, current capsules leak perfume over time and thus fail to have the required leakage profile—particularly over time at high temperatures. In such cases, the perfume is not delivered in the quantity that is desired as such perfume is no longer encapsulated. Thus, the desired effectiveness of the benefit is not obtained.

Accordingly, there is a need for an encapsulate that provides improved benefit agent delivery. Here, Applicants recognized that the source of the leakage problem was not only due to the level of cross-links between the molecules in the shell/wall of the encapsulate but was also due to the low packing density of the molecules in the shell/wall of the encapsulate. While not being bound by theory, applicants believe that the encapsulates that are disclosed herein have the correct packing density and thus meet the aforementioned need as such encapsulates are tailored such that they have the desired leakage profile.

### SUMMARY OF THE INVENTION

Encapsulates, compositions, packaged products and displays comprising such encapsulates, and processes for making and using such encapsulates, compositions, packaged products and displays are disclosed. Such encapsulates comprise a core comprising a benefit agent and a shell that at least partially surrounds said core, such encapsulates further comprise a density balancing agent.

### DETAILED DESCRIPTION OF THE INVENTION

### Definitions

As used herein “consumer product” means baby care, beauty care, fabric & home care, family care, feminine care, health care, snack and/or beverage products or devices intended to be used or consumed in the form in which it is sold, and not intended for subsequent commercial manufacture or modification. Such products include but are not limited to diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, RX pharmaceuticals, pet health and nutrition, and water purification; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); and coffee.

As used herein, the term “cleaning and/or treatment composition” includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, dentifrice, car or carpet shampoos, bathroom cleaners; hair shampoos and hair-rinses; shower gels and foam baths and metal cleaners; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists.

As used herein, the term “fabric care composition” includes, unless otherwise indicated, fabric softening compositions, fabric enhancing compositions, fabric freshening compositions and combinations there of.

As used herein, the articles “a” and “an” when used in a claim, are understood to mean one or more of what is claimed or described.

As used herein, the terms “include”, “includes” and “including” are meant to be synonymous with the phrase “including but not limited to”.

As used herein, the term “solid” means granular, powder, bar and tablet product forms.

As used herein, the term “situs” includes paper products, fabrics, garments, hard surfaces, hair and skin.

The test methods disclosed in the Test Methods Section of the present application should be used to determine the respective values of the parameters of Applicants' inventions.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Encapsulates and Compositions Comprising Same

The time period for determining the leakage profile of an encapsulate may include the time the encapsulate is in product and the time such product is in use. The satisfactory delivery of the content of an encapsulate requires optimum capsule mechanical properties as if the capsule is too strong, it never releases its content and if a capsule is too weak, it breaks to soon thus releasing it contents prematurely. In addition, capsule mechanical properties can be compromised by various factors such as prolonged exposure at high temperature and/or low pH and thus the leakage profile of a capsule with optimal mechanical properties can be compromised.

Applicants recognized that the source of the aforementioned leakage problem was not only due to the level of cross-links between the molecules in the shell/wall of the encapsulate but was also due to the low packing density of the molecules in the shell/wall of the encapsulate. Applicants not only recognized the source of the leakage profile problem but also recognized that encapsulates having the required cross-linking and packaging density can be identified and characterized by their ATR-FTIR value and/or SAXS Bump Descriptor value. Such, encapsulates and compositions comprising such encapsulates are disclosed below.

In one aspect, said encapsulate is a perfume microcapsule.

In one aspect, a composition that may comprise:
a) based on total composition weight, from about 0.001% to about 10%, from about 0.001% to about 8%, or even from about 0.01% to about 5% of an encapsulate selected from the group consisting of
   (i) an encapsulate comprising a core comprising a benefit agent and a shell that encapsulates said core, said encapsulate's shell comprising cross-linked melamine formaldehyde and having an ATR-FTIR spectrum second derivative 1490:1550 cm⁻¹ (±2 cm⁻¹) peak ratio from about 0.1 to about 0.7, from about 0.1 to about 0.5, from about 0.1 to about 0.4, from about 0.1 to about 0.3, or even from about 0.1 to about 0.2;
   (ii) an encapsulate comprising a core comprising a benefit agent and a shell that encapsulates said core, said encapsulate's shell comprising cross-linked melamine formaldehyde and having an ATR-FTIR spectrum second derivative 790:813 cm⁻¹ (±2 cm⁻¹) peak ratio from 0 to about 0.1, from 0 to about 0.08, or even from 0 to about 0.05;
   (iii) an encapsulate comprising a core comprising a benefit agent and a shell that at least encapsulates said core, said encapsulate's shell having a SAXS Bump Descriptor value from about 2 to about 1,000,000, from about 4 to about 100,000, from about 10 to about 1,000 or even from about 10 to about 100;
   (iv) an encapsulate comprising a core comprising a benefit agent and a shell that encapsulates said core, said encapsulate's shell comprising cross-linked melamine formaldehyde and having an ATR-FTIR spectrum second derivative 790:813 cm⁻¹ (±2 cm⁻¹) peak ratio from 0 to about 0.1, from 0 to about 0.08, or even from 0 to about 0.05 and a SAXS Bump Descriptor value from about 2 to about 1,000,000, from about 4 to about 100,000, from about 10 to about 1,000 or even from about 10 to about 100;
   (v) mixtures thereof;
said encapsulates having a wall thickness from about 1 nm to about 200 nm, from about 5 nm to about 200 nm, from about 20 nm to about 200 nm, from about 25 nm to about 150 nm, from about 30 nm to about 125 nm or even from about 35 nm to about 100 nm; an encapsulate wall thickness polydispersity from about 0.01 to about 0.2, from about 0.02 to about 0.1, or even from about 0.03 to about 0.08; a particle size median from about 1 micron to about 100 microns, from about 2 microns to about 60 microns, from about 3 microns to about 35 microns or even from about 5 microns to 25 microns; and at least 75%, 85%, 95% or even about 100% of said encapsulates having a fracture strength from about 0.2 MPa to about 10 MPa, from about 0.4 to about 7 MPa, from about 0.4 to about 5 MPa;
b) a material selected from the group consisting of a surfactant, a builder, a chelating agent, a dye transfer inhibiting agent, a dispersant, an enzyme, an enzyme stabilizer, a catalytic bleaching material, a bleach activator, a polymeric dispersing agent, a clay soil removal/anti-redeposition agent, a brightener, a suds suppressor, a dye, a structure elasticizing agent, a thickener/structurant, a fabric softener, a carrier, a hydrotrope, a pigment, a silicone and mixtures thereof;
   said composition being a solid detergent; a liquid detergent comprising, based on total liquid detergent weight, less than about 60% water, less than about 60% to about 2% water, from about 45% to about 7% water, from about 35% to about 9% water and having a neat viscosity of from about 10 cps to about 999 cps, or even from about 100 cps to about 800 cps; a detergent gel comprising, based on total gel weight, less than about 45% water less than about 45% to about 2% water, from about 45% to about 7% water, from about 35% to about 9% water and having a neat viscosity of from about 1,000 cps to about 10,000 cps or even from about 1,200 cps to about 8,000 cps; a fabric enhancer; a shampoo; a hair conditioner; or a unit dose detergent comprising a detergent and a water soluble film encapsulating said detergent
   is disclosed.

In one aspect of said composition, said composition may comprise based on total composition weight, from about 0.001% to about 10%, from about 0.001% to about 8%, or even from about 0.01% to about 5% of an encapsulate comprising a core comprising a benefit agent and a shell that encapsulates said core, said encapsulate's shell comprising cross-linked melamine formaldehyde and having an ATR-FTIR second derivative 790:813 cm⁻¹ (±2 cm⁻¹) peak ratio from 0 to about 0.1, from 0 to about 0.08, or even from 0 to about 0.05 and a SAXS Bump Descriptor value from about 2 to about 1,000,000, from about 4 to about 100,000, from about 10 to about 1,000 or even from about 10 to about 100.

In one aspect of said composition of said encapsulate's shell may comprise a material selected from the group consisting of polyethylenes; polyamides; polystyrenes; polyisoprenes; polycarbonates; polyesters; polyacrylates; aminoplasts, in one aspect said aminoplast comprises a polyureas, polyurethane, and/or polyureaurethane, in one aspect said polyurea comprises polyoxymethyleneurea and/or melamine formaldehyde; polyolefins; polysaccharides, in one aspect alginate and/or chitosan; gelatin; shellac; epoxy resins; vinyl polymers; water insoluble inorganics; silicone; and mixtures thereof.

In one aspect of said composition, said encapsulate's shell may comprise melamine formaldehyde and/or cross linked melamine formaldehyde.

In one aspect of said composition said encapsulate's benefit agent is selected from the group consisting of a perfume, a cooling agent, a sensate and mixtures thereof.

In one aspect of said composition, said encapsulate's core comprises perfume.

In one aspect of said composition, said encapsulate's core comprises a perfume composition selected from the group consisting of:
a) a perfume composition having a C log P of less than 4.5 to about 2, less than 4.25 to about 2.2, less than 4.0 to about 2.5 or even less than 3.75 to about 2.6;
b) a perfume composition comprising, based on total perfume composition weight, at least 60% or even at least 70% perfume materials having a C log P of less than 4.0 to about 2;
c) a perfume composition comprising, based on total perfume composition weight, at least 35%, at least 50% or even at least 60% perfume materials having a C log P of less than 3.5 to about 2;
d) a perfume composition comprising, based on total perfume composition weight, at least 40% perfume materials having a C log P of less than 4.0 to about 2 or even less than 3.5 to about 2 and at least 1% perfume materials having a C log P of less than 2.0 to about 1;
e) a perfume composition comprising, based on total perfume composition weight, at least 40% perfume materials having a C log P of less than 4.0 to about 2 or even less than 3.5 to about 2 and at least 15% perfume materials having a C log P of less than 3.0 to about 1.5;
f) a perfume composition comprising, based on total perfume composition weight, at least 1% or even at least 2% of a butanoate ester and at least 1% of a pentanoate ester;
g) a perfume composition comprising, based on total perfume composition weight, at least 2% or even at least 3% of an ester comprising an allyl moiety and at least 10%, at least 25% or even at least 30% of another perfume comprising an ester moiety;
h) a perfume composition comprising, based on total perfume composition weight, at least 1% or even at least 5% of an aldehyde comprising an alkyl chain moiety;
i) a perfume composition comprising, based on total perfume composition weight, at least 2% of a butanoate ester;
j) a perfume composition comprising, based on total perfume composition weight, at least 1% of a pentanoate ester;
k) a perfume composition comprising, based on total perfume composition weight, at least 3% of an ester comprising an allyl moiety and at least 1% of an aldehyde comprising an alkyl chain moiety; and
l) a perfume composition comprising, based on total perfume composition weight, at least 25% of a perfume comprising an ester moiety and at least 1% of an aldehyde comprising an alkyl chain.

In one aspect of said composition, said composition is a liquid detergent and said encapsulates may comprise a density balancing agent is selected from the group consisting of an organic material having a density greater than about 1, or even from greater than about 1 to about 5, an inorganic oxide, inorganic oxy-chloride, inorganic halogenide, a salt, and mixtures thereof.

In one aspect of said composition, said encapsulates may have a core to wall ratio from about 70:30 to about 98:2, from about 70:30 to about 95:5, from about 80:20 to about 93:7, or even from about 85:15 to about 90:10.

In one aspect of said composition, said encapsulate's core may comprise, based total core weight, at least 10%, at least 25%, at least 35%, at least 45% or even at least 60% of one or more Table 1 perfume raw materials.

In one aspect of said composition, said encapsulate's core may comprise a perfume that may comprise:
a) from about 3% to about 20% a perfume raw material selected from the group of Table 1 perfume raw materials 85-88, 100, 108 and mixtures thereof;
b) from about 2% to about 35% of a perfume raw material selected from the group of Table 1 perfume raw materials 62-84, 114, 115 and mixtures thereof;
c) from about 2% to about 35% of a perfume raw material selected from the group of Table 1 perfume raw materials 1-61, 101, 102, 104, 109, 113 and mixtures thereof;
d) from about 0% to about 10% of a perfume raw material selected from the group of Table 1 perfume raw materials 99, 106, 111, 112 and mixtures thereof;
e) from about 0% to about 10% of a perfume raw material selected from the group of Table 1 perfume raw materials 89-94, 107, 110 and mixtures thereof; and
f) from about 0% to about 0.5% of a perfume raw material selected from the group of Table 1 perfume raw materials 95-98, 103, 105 and mixtures thereof.

In one aspect of said composition, said encapsulate's core may comprise a perfume that may comprise:
a) from about 3% to about 10% of a perfume raw material selected from the group of Table 1 perfume raw materials 85-88, 100, 108 and mixtures thereof;
b) from about 5% to about 10% of a perfume raw material selected from the group of Table 1 perfume raw materials 62-84, 114, 115 and mixtures thereof;
c) from about 5% to about 10% of a perfume raw material selected from the group of Table 1 perfume raw materials 1-61, 101, 102, 104, 109, 113 and mixtures thereof;
d) from about 2% to about 8% of a perfume raw material selected from the group of Table 1 perfume raw materials 99, 106, 111, 112 and mixtures thereof;
e) even from about 2% to about 8% of a perfume raw material selected from the group of Table 1 perfume raw materials 89-94, 107, 110 and mixtures thereof; and
f) from about 0% to about 0.5% of a perfume raw material selected from the group of Table 1 perfume raw materials 95-98, 103, 105 and mixtures thereof.

In one aspect of said composition, said encapsulate's core may comprise a perfume that may comprise:
a) from about 3% to about 7% of a perfume raw material selected from the group of Table 1 perfume raw materials 85-88, 100, 108 and mixtures thereof;
b) from about 2.5% to about 8% of a perfume raw material selected from the group of Table 1 perfume raw materials 62-84, 114, 115 and mixtures thereof;
c) from about 5% esters to about 8% of a perfume raw material selected from the group of Table 1 perfume raw materials 1-61, 101, 102, 104, 109, 113 and mixtures thereof;
d) 2% to about 8% of a perfume raw material selected from the group of Table 1 perfume raw materials 99, 106, 111, 112 and mixtures thereof;
e) 2% to about 8% of a perfume raw material selected from the group of Table 1 perfume raw materials 89-94, 107, 110 and mixtures thereof; and
f) from about 0% to about 0.5% of a perfume raw material selected from the group of Table 1 perfume raw materials 95-98, 103, 105 and mixtures thereof.

In one aspect of said composition, said encapsulate's core may comprise a perfume that may comprise:
a) from about 3% to about 20%, from about 3% to about 10%, or even from about 3% to about 7% of a perfume raw material selected from the group of Table 1 perfume raw materials 87, 100, 108 and mixtures thereof;
b) from about 2% to about 35% of a perfume raw material selected from the group of Table 1 perfume raw materials 62-64, 66, 76, 114, 115 and mixtures thereof;
c) from about 2% to about 35% of a perfume raw material selected from the group of Table 1 perfume raw materials 2-4, 11, 49, 91 and mixtures thereof;
d) from about 0% to about 10% of a perfume raw material selected from the group of Table 1 perfume raw materials 99, 106, 111, 112 and mixtures thereof;
e) from about 0% to about 10% of a perfume raw material selected from the group of Table 1 perfume raw materials 89-94, 107, 110 and mixtures thereof; and
f) from about 0% to about 0.5% of a perfume raw material selected from the group of Table 1 perfume raw materials 95-98, 103, 105 and mixtures thereof.

In one aspect of said composition, said encapsulate' score may comprise a perfume that may comprise:
a) from about 3% to about 20% of a perfume raw material selected from the group of Table 1 perfume raw materials 87, 100, 108 and mixtures thereof;
b) from about 2% to about 35% of a perfume raw material selected from the group of Table 1 perfume raw materials 114, 115 and mixtures thereof;
c) from about 2% to about 35% of a perfume raw material selected from the group of Table 1 perfume raw materials 2-4, 11, 49, 91 and mixtures thereof;
d) from about 0% to about 10% of a perfume raw material selected from the group of Table 1 perfume raw materials 99, 106, 111, 112 and mixtures thereof;
e) from about 0% to about 10% of a perfume raw material selected from the group of Table 1 perfume raw materials 89-94, 107, 110 and mixtures thereof; and
f) from about 0% to about 0.5% of a perfume raw material selected from the group of Table 1 perfume raw materials 95-98, 103, 105 and mixtures thereof.

### Suitable Perfume Raw Materials

Perfumes that provide improved perfume performance under high soil conditions and in cold water may comprise Perfume Raw Materials as given in Table 1 below.

TABLE 1
Useful Perfume Raw Materials

| Item | Common Name | IUPAC Name |
|---|---|---|
| 1 | Methyl 2-methyl butyrate | methyl 2-methylbutanoate |
| 2 | Isopropyl 2-methyl butyrate | propan-2-yl 2-methylbutanoate |
| 3 | Ethyl-2 Methyl Butyrate | ethyl 2-methylbutanoate |
| 4 | Ethyl-2 Methyl Pentanoate | ethyl 2-methylpentanoate |
| 5 | Ethyl heptanoate | ethyl heptanoate |
| 6 | Ethyl octanoate | Ethyl octanoate |
| 7 | isobutyl hexanoate | 2-methylpropyl hexanoate |
| 8 | Amyl butyrate | pentyl butanoate |
| 9 | Amyl heptanoate | Pentyl heptanoate |
| 10 | Isoamyl isobutyrate | 3-methylbutyl 2-methylpropanoate |
| 11 | Hexyl acetate | hexyl acetate |
| 12 | hexyl butyrate | hexyl butanoate |
| 13 | hexyl isobutyrate | hexyl 2-methylpropanoate |
| 14 | hexyl isovalerate | hexyl 3-methylbutanoate |
| 15 | hexyl propionate | hexyl propanoate |
| 16 | Ethyl 2-cyclohexyl propanoate | ethyl 2-cyclohexylpropanoate |
| 17 | Ethyl 3,5,5-trimethyl hexanoate | ethyl 3,5,5-trimethylhexanoate |
| 18 | glyceryl 5-hydroxydecanoate | 2,3-dihydroxypropyl 5-hydroxydecanoate |
| 19 | Prenyl acetate | 3-methyl 2-butenyl acetate |
| 20 | 3-methyl 2-butenyl acetate | 3-methyl 2-butenyl acetate |
| 21 | methyl 3-nonenoate | methyl non-3-enoate |
| 22 | Ethyl (E)-dec-4-enoate | Ethyl (E)-dec-4-enoate |
| 23 | Ethyl (E)-oct-2-enoate | Ethyl (E)-oct-2-enoate |
| 24 | Ethyl 2,4-decadienoate | ethyl (2E,4Z)-deca-2,4-dienoate |
| 25 | Ethyl 3-octenoate | ethyl (E)-oct-3-enoate |
| 26 | Citronellyl acetate | 3,7-dimethyloct-6-enyl acetate |
| 27 | Ethyl trans-2-decenoate | ethyl (E)-dec-2-enoate |
| 28 | 2-hexen-1-yl isovalerate | [(E)-hex-2-enyl] acetate |
| 29 | 2-hexen-1-yl propionate | [(E)-hex-2-enyl] propanoate |
| 30 | 2-hexen-1-yl valerate | [(E)-hex-2-enyl] pentanoate |
| 31 | 3-hexen-1-yl (E)-2-hexenoate | [(Z)-hex-3-enyl] (E)-hex-2-enoate |
| 32 | 3-Hexen-1-yl 2-methyl butyrate | [(Z)-hex-3-enyl] 2-methylbutanoate |
| 33 | 3-hexen-1-yl acetate | [(Z)-hex-3-enyl] acetate |
| 34 | 3-hexen-1-yl benzoate | [(Z)-hex-3-enyl] benzoate |
| 35 | 3-hexen-1-yl formate | [(Z)-hex-3-enyl] formate |
| 36 | 3-hexen-1-yl tiglate | [(Z)-hex-3-enyl] (Z)-2-methylbut-2-enoate |
| 37 | 2-methyl butyl 2-methyl butyrate | 2-methylbutyl 2-methylbutanoate |
| 38 | Butyl isovalerate | butyl 3-methylbutanoate |
| 39 | Geranyl acetate | [(2E)-3,7-dimethylocta-2,6-dienyl] acetate |
| 40 | Geranyl butyrate | [(2E)-3,7-dimethylocta-2,6-dienyl] butanoate |
| 41 | Geranyl isovalerate | [(3E)-3,7-dimethylocta-3,6-dienyl] 3-methylbutanoate |
| 42 | Geranyl propionate | [(2E)-3,7-dimethylocta-2,6-dienyl] propanoate |
| 43 | Allyl cyclohexane acetate | prop-2-enyl 2-cyclohexylacetate |
| 44 | Allyl Cyclohexyl Propionate | prop-2-enyl 3-cyclohexylpropanoate |
| 45 | allyl cyclohexyl valerate | prop-2-enyl 5-cyclohexylpentanoate |
| 46 | benzyl octanoate | benzyl octanoate |
| 47 | Cocolactone | 6-pentyl-5,6-dihydropyran-2-one |
| 48 | coconut decanone | 8-methyl-1-oxaspiro(4.5)decan-2-one |
| 49 | gamma undecalactone | 5-heptyloxolan-2-one |
| 50 | gamma-decalactone | 5-hexyloxolan-2-one |
| 51 | gamma-dodecalactone | 5-octyloxolan-2-one |
| 52 | jasmin lactone | 6-[(E)-pent-2-enyl] oxan-2-one |
| 53 | Jasmolactone | 5-[(Z)-hex-3-enyl]oxolan-2-one |
| 54 | Nonalactone | 6-butyloxan-2-one |
| 55 | 6-acetoxydihydrotheaspirane | [2a,5a(S*)]-2,6,10,10-tetramethyl-1- |
| | | oxaspiro[4.5]decan-6-yl acetate |
| 56 | Phenoxyethyl isobutyrate | 2-(phenoxy)ethyl 2-methylpropanoate |
| 57 | Pivacyclene | |
| 58 | Verdox | (2-tert-butylcyclohexyl) acetate |
| 59 | Cyclobutanate | 3a,4,5,6,7,7a-hexahydro-4,7-methano-1g- |
| | | inden-5(or 6)-yl butyrate |
| 60 | Dimethyl Anthranilate | methyl 2-methylaminobenzoate |
| 61 | Methyl Antranilate | methyl 2-aminobenzoate |
| 62 | Octyl Aldehyde | Octanal |
| 63 | Nonanal | Nonanal |
| 64 | Decyl aldehyde | Decanal |
| 65 | Lauric Aldehyde | Dodecanal |
| 66 | Methyl Nonyl Acetaldehyde | 2-methyl undecanal |
| 67 | Methyl Octyl Acetaldehyde | 2-methyl decanal |
| 68 | 2,4-Hexadienal | (2E,4E)-hexa-2,4-dienal |
| 69 | Intreleven Aldehyde | undec-10-enal |
| 70 | Decen-1-al | (E)-dec-2-enal |
| 71 | Nonen-1-al | (E)-2-nonen-1-al |
| 72 | Adoxal | 2,6,10-trimethylundec-9-enal |
| 73 | Geraldehyde | (4Z)-5,9-dimethyldeca-4,8-dienal |
| 74 | Iso cyclo citral | 2,4,6-trimethylcyclohex-3-ene-1-carbaldehyde |
| 75 | d-limonene mainly | 1-methyl-4-prop-1-en-2-yl-cyclohexene |
| 76 | Ligustral | 2,4-dimethylcyclohex-3-ene-1-carbaldehyde |
| 77 | Myrac aldehyde | 4-(4-methylpent-3-enyl)cyclohex-3-ene-1-carbaldehyde |
| 78 | Tridecenal | tridec-2-enal |
| 79 | Triplal | 2,4-dimethyl-3-cyclohexene-1-carboxaldehyde |
| 80 | Vertoliff | 1,2-dimethylcyclohex-3-ene-1-carbaldehyde |
| 81 | Cyclal C | 2,4-dimethylcyclohex-3-ene-1-carbaldehyde |
| 82 | Anisic aldehyde | 4-methoxybenzaldehyde |
| 83 | Helional | 3-(1,3-benzodioxol-5-yl)-2-methylpropanal |
| 84 | Heliotropin | 1,3-benzodioxole-5-carbaldehyde |
| 85 | Neocaspirene | |
| 86 | Beta Naphthol Ethyl Ether | 2-ethoxynaphtalene |
| 87 | Beta Naphthol Methyl Ether | 2-methoxynaphtalene |
| 88 | hyacinth ether | 2-cyclohexyloxyethylbenzene |
| 89 | 2-heptyl cyclopentanone (fleuramone) | 2-heptylcyclopentan-1-one |
| 90 | menthone-8-thioacetate | O-[2-[(1S)-4-methyl-2- |
| | | oxocyclohexyl]propan-2-yl] ethanethioate |
| 91 | Nectaryl | 2-[2-(4-methyl-1-cyclohex-3- |
| | | enyl)propyl]cyclopentan-1-one |
| 92 | Phenyl Naphthyl Ketone | naphthalen-2-yl-phenylmethanone |
| 93 | decen-1-yl cyclopentanone | 2-[(2E)-3,7-dimethylocta-2,6-dienyl] |
| | | cyclopentan-1-one |
| 94 | fruity cyclopentanone (veloutone) | 2,2,5-trimethyl-5-pentylcyclopentan-1-one |
| 95 | 4-methoxy-2-methyl butane thiol | 4-methoxy-2-methylbutane-2-thiol |
| | (blackcurrant mercaptan) | |
| 96 | Grapefruit Mercaptan | 2-(4-methyl-1-cyclohex-3-enyl)propane-2-thiol |
| 97 | Buccoxime | N-(1,5-dimethyl-8- |
| | | bicyclo[3.2.1]octanylidene)hydroxylamine |
| 98 | Labienoxime | 2,4,4,7-Tetramethyl-6,8-nonadiene-3-one oxime |
| 99 | Undecavertol | (E)-4-methyldec-3-en-5-ol |
| 100 | Decanal diethyl acetal | 1,1-diethoxydecane |
| 101 | Diethyl maleate | diethyl but-2-enedioate |
| 102 | Ethyl Acetoacetate | ethyl 3-oxobutanoate |
| 103 | frutonile | 2-Methyldecanenitrile |
| 104 | Methyl dioxolan | ethyl 2-(2-methyl-1,3-dioxolan-2-yl)acetate |
| 105 | Cetalox | 3a,6,6,9a-tetramethyl-2,4,5,5a,7,8,9,9b- |
| | | octahydro-1H-benzo[e][1]benzofuran |
| 106 | Cyclopentol | |
| 107 | Delta-damascone | (E)-1-(2,6,6-trimethyl-1-cyclohex-3- |
| | | enyl)but-2-en-1-one |
| 108 | Eucalyptol | 1,3,3-trimethyl-2-oxabicyclo[2,2,2]octane |
| 109 | Flor acetate | |
| 110 | Ionone gamma methyl | (E)-3-methyl-4-(2,6,6-trimethyl-1- |
| | | cyclohex-2-enyl)but-3-en-2-one |
| 111 | Laevo trisandol | |
| 112 | Linalool | 3,7-dimethylocta-1,6-dien-3-ol |
| 113 | Violiff | [(4Z)-1-cyclooct-4-enyl] methyl carbonate |
| 114 | Cymal | 3-(4-propan-2-ylphenyl)butanal |
| 115 | Bourgeonal | 3-(4-tert-butylphenyl)propanal |

In one aspect of said composition, said composition may comprise any of the encapsulates described herein and have any of the parameters disclosed herein

### Process of Making Encapsulates

The encapsulates disclosed in the present specification may be made in accordance with the examples of the present specification and the following teachings:

In one aspect, said encapsulates may be made by a process that may comprise:
a) preparing a first solution comprising, based on total solution weight from about 20% to about 90%, from about 40% to about 80%, or even from about 60% to about 80% water, a first emulsifier (can be mixtures of emulsifiers) and a first resin, the ratio of said first emulsifier and said first resin being from about from about 1:10 to about 10:1, from about 1:6 to about 4:1, or even from about 1:4 to about 3:1;
b) preparing a second solution comprising based on total solution weight from about 20% to about 95% water, a second emulsifier and a second resin, the ratio of said second emulsifier and said second resin being from about 1:100 to about 10:1, from about 1:30 to about 4:1, or even from about 1:10 to about 2:1;
c) combining a core material and said first solution to form a first composition;
d) emulsifying said first composition;
e) for the first and second solution the pH is adjusted from about 3 to about 7, from about 4 to about 6.5, or even from about 5 to about 6;
f) for the first solution the temperature of operation is from about 40° C. to about 90° C., from about 50° C. to about 80° C., from about 55° C. to about 70° C.;
g) for the second solution the temperature of operation is from about 5° C. to about 50° C., from about 10° C. to about 40° C., from about 15° C. to about 30° C.;
h) combining said first composition and said second solution to form a second composition and optionally combining any processing aids and said second composition—said first composition and said second solution may be combined in any order but in one aspect said second solution is added to said first composition or said second solution and said first composition are combined simultaneously;
i) mixing said second composition for at least 15 minutes, at least 1 hour or even from about 4 hours to about 100 hours at a temperature of from about 25° C. to about 100° C., from about 45° C. to about 90° C., or even from about 50° C. to about 85° C. and optionally combining any processing aids to said second composition;
j) optionally combining any scavenger material, structurant, and/or anti-agglomeration agent with said second composition during step or thereafter—such materials may be combined in any order but in one aspect the scavenger material is combined first, any structurant second, and then anti-agglomeration agent is combined; and
k) optionally spray drying said or agglomeration of the second composition is disclosed.

Suitable equipment for use in the processes disclosed herein may include continuous stirred tank reactors, homogenizers, turbine agitators, recirculating pumps, paddle mixers, ploughshear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

### Adjunct Materials

While not essential for each consumer product embodiment of the present invention, the non-limiting list of adjuncts illustrated hereinafter are suitable for use in the instant consumer products and may be desirably incorporated in certain embodiments of the invention, for example to assist or enhance performance, for treatment of the substrate to be cleaned, or to modify the aesthetics of the composition as is the case with perfumes, colorants, dyes or the like. The precise nature of these additional components, and levels of incorporation thereof, will depend on the physical form of the composition and the nature of the operation for which it is to be used. Suitable adjunct materials include, but are not limited to, surfactants, builders, chelating agents, dye transfer inhibiting agents, dispersants, enzymes, and enzyme stabilizers, catalytic materials, bleach activators, polymeric dispersing agents, clay soil removal/anti-redeposition agents, brighteners, suds suppressors, dyes, additional perfume and perfume delivery systems, structure elasticizing agents, thickeners/structurants, fabric softeners, carriers, hydrotropes, processing aids and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the adjunct ingredients are not essential for each consumer product embodiment of the present invention. Thus, certain embodiments of Applicants' compositions do not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure elasticizing agents, thickeners/structurants, fabric softeners, carriers, hydrotropes, processing aids and/or pigments. However, when one or more adjuncts is present, such one or more adjuncts may be present as detailed below:

Surfactants—The compositions according to the present invention can comprise a surfactant or surfactant system wherein the surfactant can be selected from nonionic and/or anionic and/or cationic surfactants and/or ampholytic and/or zwitterionic and/or semi-polar nonionic surfactants. The surfactant is typically present at a level of from about 0.1%, from about 1%, or even from about 5% by weight of the cleaning compositions to about 99.9%, to about 80%, to about 35%, or even to about 30% by weight of the cleaning compositions.

Builders—The compositions of the present invention can comprise one or more detergent builders or builder systems. When present, the compositions will typically comprise at least about 1% builder, or from about 5% or 10% to about 80%, 50%, or even 30% by weight, of said builder. Builders include, but are not limited to, the alkali metal, ammonium and alkanolammonium salts of polyphosphates, alkali metal silicates, alkaline earth and alkali metal carbonates, aluminosilicate builders polycarboxylate compounds. ether hydroxypolycarboxylates, copolymers of maleic anhydride with ethylene or vinyl methyl ether, 1,3,5-trihydroxybenzene-2,4,6-trisulphonic acid, and carboxymethyl-oxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Chelating Agents—The compositions herein may also optionally contain one or more copper, iron and/or manganese chelating agents. If utilized, chelating agents will generally comprise from about 0.1% by weight of the compositions herein to about 15%, or even from about 3.0% to about 15% by weight of the compositions herein.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in the compositions herein, the dye transfer inhibiting agents are present at levels from about 0.0001%, from about 0.01%, from about 0.05% by weight of the cleaning compositions to about 10%, about 2%, or even about 1% by weight of the cleaning compositions.

Dispersants—The compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The compositions can comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is a cocktail of conventional applicable enzymes like protease, lipase, cutinase and/or cellulase in conjunction with amylase.

Enzyme Stabilizers—Enzymes for use in compositions, for example, detergents can be stabilized by various techniques. The enzymes employed herein can be stabilized by the presence of water-soluble sources of calcium and/or magnesium ions in the finished compositions that provide such ions to the enzymes.

Catalytic Metal Complexes—Applicants' compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methyl-enephosphonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, for example, the manganese-based catalysts disclosed in U.S. Pat. No. 5,576,282.

Cobalt bleach catalysts useful herein are known, and are described, for example, in U.S. Pat. Nos. 5,597,936 and 5,595,967. Such cobalt catalysts are readily prepared by known procedures, such as taught for example in U.S. Pat. Nos. 5,597,936, and 5,595,967.

Compositions herein may also suitably include a transition metal complex of a macropolycyclic rigid ligand—abbreviated as “MRL”. As a practical matter, and not by way of limitation, the compositions and cleaning processes herein can be adjusted to provide on the order of at least one part per hundred million of the benefit agent MRL species in the aqueous washing medium, and may provide from about 0.005 ppm to about 25 ppm, from about 0.05 ppm to about 10 ppm, or even from about 0.1 ppm to about 5 ppm, of the MRL in the wash liquor.

Preferred transition-metals in the instant transition-metal bleach catalyst include manganese, iron and chromium. Preferred MRL's herein are a special type of ultra-rigid ligand that is cross-bridged such as 5,12-diethyl-1,5,8,12-tetraazabicyclo[6.6.2]hexa-decane.

Suitable transition metal MRLs are readily prepared by known procedures, such as taught for example in WO 00/32601, and U.S. Pat. No. 6,225,464.

Suitable thickeners/structurants and useful levels of same are described in U.S. Patent Application Publication No. 2005/0130864 A1 and U.S. Pat. Nos. 7,169,741 B2 and 7,297,674 B2. In one aspect, the thickner may be a rheology modifier. The rheology modifier may be selected from the group consisting of non-polymeric crystalline, hydroxy-functional materials, polymeric rheology modifiers which impart shear thinning characteristics to the aqueous liquid matrix of the composition. In one aspect, such rheology modifiers impart to the aqueous liquid composition a high shear viscosity, at 20 sec⁻¹ shear rate and at 21° C., of from 1 to 7000 cps and a viscosity at low shear (0.5 sec⁻¹ shear rate at 21° C.) of greater than 1000 cps, or even 1000 cps to 200,000 cps. In one aspect, for cleaning and treatment compositions, such rheology modifiers impart to the aqueous liquid composition a high shear viscosity, at 20 sec⁻¹ and at 21° C., of from 50 to 3000 cps and a viscosity at low shear (0.5 sec⁻¹ shear rate at 21° C.) of greater than 1000 cps, or even 1000 cps to 200,000 cps. Viscosity according to the present invention is measured using an AR 2000 rheometer from TA instruments using a plate steel spindle having a plate diameter of 40 mm and a gap size of 500 μm. The high shear viscosity at 20 sec⁻¹ and low shear viscosity at 0.5 sec⁻¹ can be obtained from a logarithmic shear rate sweep from 0.1 sec⁻¹ to 25 sec⁻¹ in 3 minutes time at 21° C. Crystalline hydroxyl functional materials are rheology modifiers which form thread-like structuring systems throughout the matrix of the composition upon in situ crystallization in the matrix. Polymeric rheology modifiers are preferably selected from polyacrylates, polymeric gums, other non-gum polysaccharides, and combinations of these polymeric materials.

Generally the rheology modifier will comprise from 0.01% to 1% by weight, preferably from 0.05% to 0.75% by weight, more preferably from 0.1% to 0.5% by weight, of the compositions herein.

Structuring agent which are especially useful in the compositions of the present invention comprises non-polymeric (except for conventional alkoxylation), crystalline hydroxy-functional materials which can form thread-like structuring systems throughout the liquid matrix when they are crystallized within the matrix in situ. Such materials can be generally characterized as crystalline, hydroxyl-containing fatty acids, fatty esters or fatty waxes. In one aspect, rheology modifiers include crystalline, hydroxyl-containing rheology modifiers include castor oil and its derivatives. In one aspect, rheology modifiers include may be hydrogenated castor oil derivatives such as hydrogenated castor oil and hydrogenated castor wax. Commercially available, castor oil-based, crystalline, hydroxyl-containing rheology modifiers include THIXCIN™ from Rheox, Inc. (now Elementis).

Other types of rheology modifiers, besides the non-polymeric, crystalline, hydroxyl-containing rheology modifiers described heretofore, may be utilized in the liquid detergent compositions herein. Polymeric materials which provide shear-thinning characteristics to the aqueous liquid matrix may also be employed.

Suitable polymeric rheology modifiers include those of the polyacrylate, polysaccharide or polysaccharide derivative type. Polysaccharide derivatives typically used as rheology modifiers comprise polymeric gum materials. Such gums include pectine, alginate, arabinogalactan (gum Arabic), carrageenan, gellan gum, xanthan gum and guar gum.

If polymeric rheology modifiers are employed herein, a preferred material of this type is gellan gum. Gellan gum is a heteropolysaccharide prepared by fermentation of Pseudomonaselodea ATCC 31461. Gellan gum is commercially marketed by CP Kelco U.S., Inc. under the KELCOGEL tradename.

A further alternative and suitable rheology modifier include a combination of a solvent and a polycarboxylate polymer. More specifically the solvent may be an alkylene glycol. In one aspect, the solvent may compriser dipropylene glycol. In one aspect, the polycarboxylate polymer may comprise a polyacrylate, polymethacrylate or mixtures thereof. In one aspect, solvent may be present, based on total composition weight, at a level of from 0.5% to 15%, or from 2% to 9% of the composition. In one aspect, polycarboxylate polymer may be present, based on total composition weight, at a level of from 0.1% to 10%, or from 2% to 5%. In one aspect, the may comprise mixture of dipropylene glycol and 1,2-propanediol. In one solvent component aspect, the ratio of dipropylene glycol to 1,2-propanediol may be 3:1 to 1:3, or even 1:1. In one aspect, the polyacrylate may comprise a copolymer of unsaturated mono- or di-carbonic acid and C₁-C₃₀ alkyl ester of the (meth) acrylic acid. In another aspect, the rheology modifier may comprise a polyacrylate of unsaturated mono- or di-carbonic acid and C₁-C₃₀ alkyl ester of the (meth) acrylic acid. Such copolymers are available from Noveon Inc under the tradename Carbopol Aqua 30®. In the absence of rheology modifier and in order to impart the desired shear thinning characteristics to the liquid composition, the liquid composition can be internally structured through surfactant phase chemistry or gel phases.

### Processes of Making and Using Compositions

The embodiments of the compositions of the present invention can be formulated into any suitable form and prepared by any process chosen by the formulator, non-limiting examples of which are described in U.S. Pat. No. 5,879,584; U.S. Pat. No. 5,691,297; U.S. Pat. No. 5,574,005; U.S. Pat. No. 5,569,645; U.S. Pat. No. 5,565,422; U.S. Pat. No. 5,516,448; U.S. Pat. No. 5,489,392; U.S. Pat. No. 5,486,303 all of which are incorporated herein by reference.

### Method of Use

Compositions disclosed herein that contain the encapsulate disclosed herein can be used to clean or treat a situs inter alia a surface or fabric. Typically at least a portion of the situs is contacted with an embodiment of Applicants' composition, in neat form or diluted in a liquor, for example, a wash liquor and then the situs may be optionally washed and/or rinsed. In one aspect, a situs is optionally washed and/or rinsed, contacted with a encapsulate according to the present invention or composition comprising said encapsulate and then optionally washed and/or rinsed. For purposes of the present invention, washing includes but is not limited to, scrubbing, and mechanical agitation. The situs may comprise most any material, for example a fabric, fabric capable of being laundered or treated in normal consumer use conditions. Liquors that may comprise the disclosed compositions may have a pH of from about 3 to about 11.5. Such compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric ratio is typically from about 1:1 to about 30:1.

### Test Methods

It is understood that the test methods that are disclosed in the Test Methods Section of the present application are used to determine the respective values of the parameters of Applicants' invention as such invention is described and claimed herein.

### Sample Preparation for Test Methods

Before the PMC slurries can be used for the described tests, the sample is homogenized by shaking the sample for 20 minutes on a shaking table such as the Heidolph Promax 2020. Once homogenized, a 200 ml glass jar is filled with the slurry. This glass jar is then put on storage for the required time and condition. After the storage period, each 200 ml sample is again homogenized for 20 minutes on the shaking table. After homogenization the slurry is used for the experiments.

In case of finished product making, the needed amount of slurry is sampled directly from the 200 ml glass jar. When the slurry is submitted for fracture strength, static smudge, SAXS or ATR-FTIR measurements, 30 g of the homogenized slurry is added into a glass tube.

### Storage Conditions:

Slurry Storage Time: 2 weeks, 3 weeks, 1 month, 3 months, 5 months, 6 months, 7 months and 9 months±2 days, Temperature 20° C., 30° C., 35° C., 40° C. and 43° C.±1° C., and pH 4.5, 5.2, 5.6 and 5.8±0.2.

Finished Product Containing Encapsulates: Time 1 week, 3 weeks, 1 month, 3 months, 6 months, 9 months and 12 months±2 days, Temperature 20° C., 30° C., 35° C., 40° C. and 43° C.±1° C., and pH 3.0, 3.5, 4.0, 7.0, 7.4 and 8.0±0.2

Application Conditions: During the wash cycle, on the wet fabric situs, on the dry fabric hair situs.

### Static Smudge Test

General Principle: The “Static Smudge” is a test method aiming to determine the percentage of encapsulated perfume oil that is released from micro-capsules under well-defined pressure conditions.

### Methodology & Instrumentation

the test method makes use of the industry standard “Mullen Burst Tester” for the application of well-controlled pressure on the PMC.
the analytical determination is a two-steps measure of the percentage of perfume oil released from the capsules after carrying out the test.

### Sample Preparation

The PMC slurry (capsule activity ˜30%) is homogenized first manually for 1 minute, and after that for 30 minutes using a rotary shaker. Next, an aqueous dilution of the homogenized slurry is prepared (˜100 mg slurry in 20 ml water). The solution is again first mixed manually for 15 seconds and then further using a rotary shaker for 1 hour in order to dissolve all non-PMC residue.

An aliquot of the PMC aqueous dilution is filtered on a membrane (SMWP 5.0 um from Millipore cat no. SMWP02500) to separate the PMCs from the rest of the material. The membrane is covered by an untreated similar membrane, placed on a modified Mullen-Tester (Standex Company). The instrument applies then a pressure (e.g. 100 psi or 200 psi) for 30 seconds. The two membranes are then first treated during 15 minutes with hexane which enables the extraction of the released perfume from broken or damaged capsules. Secondly, the membranes are transferred to a methanolic phase which upon heat treatment (30 minutes at 60° C.) allows to release the remaining perfume oil kept into the intact capsules. The perfume oil level is quantitatively determined in both fractions via Mass Spectrometry (LC-MS/MS Sciex Applied Biosystems API3000) using an ISTD external calibration method.

### Calculation of Percentage Oil Released

The sum of the perfume oil content in the hexane phase and in the methanol phase corresponds to the total encapsulated oil level in the PMC. The ratio between the oil content in the hexane phase and the total oil level is defined as the percentage of Oil Released of a PMC batch.

### ATR-FTIR Method

### Sample Preparation

5 mL of PMC slurry in a 50 mL conical-bottom polypropylene centrifuge tube is dispersed with 25 mL MQ water and shaken vigorously. The solution is centrifuged for 10 minutes at 9200 RPM, 20° C. The PMCs containing perfume form a low density layer on top of the aqueous solution; this layer is transferred to another 50 mL conical centrifuge and dispersed again with 25 mL MQ water. The solution is centrifuged again at 9200 RPM, 20° C. The water is removed with a plastic transfer pipette. The water cleaned PMC slurry is dispersed in 25 mL methanol and the solution is shaken in the tube for 5 minutes using the mechanical shaking hand. The solution is centrifuged for 10 minutes at 9200 RPM, 20° C. PMCs without perfume precipitate at the bottom of the tube and the perfume dissolved in methanol is decanted. The process of methanol dispersion, shaking, centrifugation, and decantation is repeated at least 3 times. The PMC slurry is suspended in water to remove the remaining methanol, shaken and centrifuged. The water is decanted. Finally, PMCs are freeze dried by dispersing PMCs in ˜20 mL MQ water, freezing the solution with liquid nitrogen and placed in the freeze dryer for ˜3 days. Dry PMC powder for ATR-FTIR analysis is obtained.

### ATR-FTIR Test

ATR-FTIR analysis is performed by placing and pressing a small amount of PMC powder on top of a germanium internal reflection element (IRE) in a Silver Gate ATR accessory (SPECAC) attached to a Perkin Elmer Spectrum One FTIR spectrometer. Spectra are collected using 80 to 128 co-added scans at a resolution of 4 cm⁻¹. Spectral analysis is performed using Thermo GRAMS/32 third-party software. Second-order derivatization of ATR-FTIR spectra is performed using the Savitsky-Golay function (25 points). The peak intensity ratios of the peaks 1490 cm⁻¹±2 cm⁻¹ and 1550 cm⁻¹±2 cm⁻¹ (1490:1550 ratio), and 790 cm⁻¹+2 cm⁻¹ and 813 cm⁻¹+2 cm⁻¹ (790:813 ratio) are calculated and reported.

### SAXS Bump Descriptor Value Method—Small Angle X-Ray Scattering Experimental Procedure

### Instrument Set-Up

SAXS measurements are carried out with a HECUS SWAX-camera (Kratky) equipped with a position-sensitive detector (OED 50M) containing 1024 channels of width 54 μm. Cu K_{α} radiation of wavelength, λ=1.542 Å, is provided by a Seifert ID-3003 X-ray generator (sealed-tube type), operating at a maximum power of 2 kW. A 10 μm thick Ni-filter is used to remove the Cu K_{α} radiation. The sample-to-detector distance is 275 mm. The volume between the sample and the detector is kept under vacuum during the measurements to minimize scattering from the air. The Kratky camera is calibrated in the small angle region using silver behenate (d=58.38 Å). Scattering curves are obtained in the Q-range, Q=4π sin θ/λ, between 0.009 and 0.54 Å⁻¹, Q being the scattering vector, and 2θ the scattering angle. Samples are filled either into a 1 mm quartz capillary or into a 1 mm demountable cell having Kapton films as windows. Standard measurement conditions are 40 kV, 20 mA and 3 hr (acquisition time). The intensities of the sample and the water/cell are divided by the actual instrumental power (voltage and amperage i.e. 40 kV and 20 mA gives 800 as dividing factor) and by the total measuring time in seconds.

### Test to Identify Efficient Capsules

### Model Fitting

In order to discriminate efficient and non-efficient capsules qualitative observations can be done. The evidence comes directly from the plot profile of the SAXS experiment: “bumps” are always present in good capsules' profiles and absent in the profiles of leaking capsules.

The analytical scattering function can be derived for particles of known shapes like sphere, circular disc, thin rod etc. The model function is then used to interpolate experimental SAXS profiles I(Q) vs Q thus obtaining structural information on scattering objects.

The model used to fit our experimental curves is the “poly core-shell ratio” [Hayter, J. B. in “Physics of Amphiphiles-Micelles, Vescicles and Microemulsions” Eds. V. DeGiorgio, M. Corti, 1983, 59-93, eqs: 32-37].

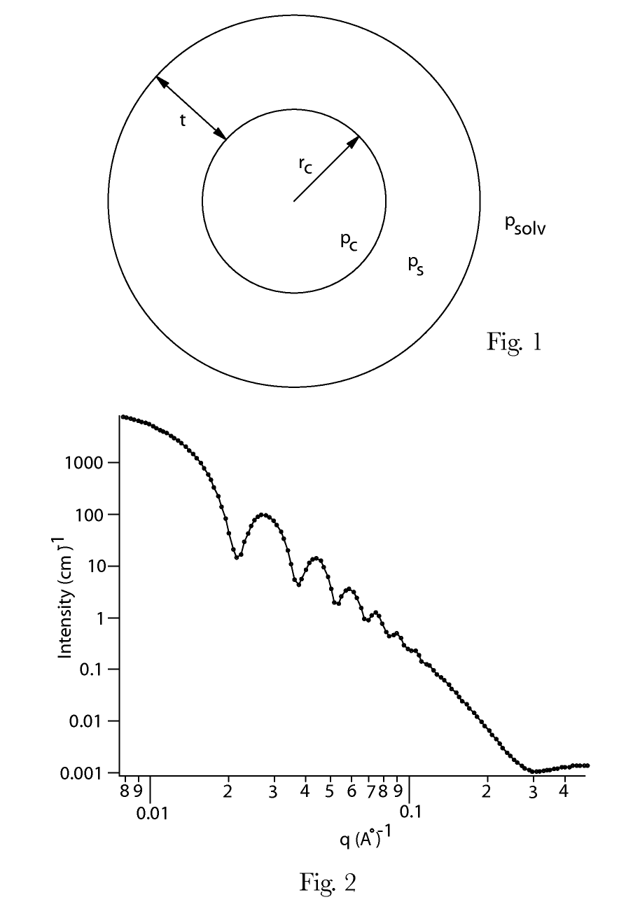

The sketch of a core-shell particle is shown in FIG. 1 and a typical core-shell profile is shown in FIG. 2, while in Table 1 the fitting parameters are reported.

r_{c} is the core radius, t is the shell thickness, r=r_{c}+t, Vₚ is the overall droplet volume, and ρ_{core}, ρₛₕₑₗₗ and ρₛₒₗᵥ are the scattering length densities of core, shell and solvent (water), respectively.

TABLE 1
Fitting parameters for core-shell model

| Models Parameters | Fitting values |
|---|---|
| Scale | 1 |
| average core radius, r_{c} (Å) | 200 |
| average shell thickness, t (Å) | 10 |
| overall polydispersity, PD | 0.05 |
| SLD core, ρ_{core} (Å⁻²) | 1E−06 |
| SLD shell, ρₛₕₑₗₗ (Å⁻²) | 2E−06 |
| SLD solvent, ρₛₒₗᵥ (Å⁻²) | 3E−06 |
| bkg (cm⁻¹) | 0.001 |

### Calculation of Wall Thickness Polydispersity

SAXS data as obtained by the HECUS instrument are first desmeared according to the Lake or Singh procedure (3D-View package is included along with the software of the instrument). Before proceeding to the fitting, Scattering Length Densities, SLD, need to be calculated, according to the following equation.

$$S L {D}_{j} = \frac{{\sum }_{i = 1}^{n} {b}_{i}}{{v}_{m}} \qquad Equation 1$$

where bᵢ is the X-ray scattering length of the i-th atom in the pure compound constituting the j-th phase (i.e. core, wall, dispersing medium) and vᵢₙ is the molecular volume.

This calculation can be performed by using the scattering length density calculator available as a Java applet present in the webpage: http://www.ncnnnist.gov/resources/sldcalc.html

In case of complex (non pure) phases, the overall phase SLDⱼ is obtained as the volume weighed mean of SLDs of phase components.

$$S L {D}_{j} = {\sum }_{k = 1}^{compounds in phase j} {x}_{k} \cdot S L {D}_{k} \qquad Equation 2$$

where xₖ is the volume fraction of k-th compound in j-th phase.

SLDs calculated for analyzed samples are reported in Table 2.

TABLE 2
Scattering length densities calculated for analyzed samples

| Pure compounds or mixed phases | SLD values (Å⁻²) |
|---|---|
| H₂O | 9.46E−06 |
| D₂O | 9.4E−06 |
| Hydrogenated o-xylene | 8.18E−06 |
| Dueuterated o-xylene | 8.09E−06 |
| Melamine-formaldheyde | 1.01E−05 |
| Core (several perfume raw materials) | 8.79E−06 |
| Solvent (H₂O + scavenger + stabilizer + MgCl₂) | 9.346E−06 |

Once the appropriate SLDs are calculated and chosen, the fitting is performed using scale, t, PD and bkg as free variables, while r_{c}, ρ_{core}, ρₛₕₑₗₗ and ρₛₒₗᵥ are kept fixed. PD values must be constrained between 0 and 1, in order to avoid physically meaningless values. The non-linear least square approach contained in Igor Pro 6 is used to reach convergence (i.e. minimum value of chi-squared, where the error bars on I-values are used as weight).

The physical information obtained from this analysis are:

I. Core radius

II. Shell radius

III. Polydispersity

I. Core radius (i.e. the whole capsule inner radius). This parameter cannot be accessed by SAXS since capsule dimension is greater than the maximum dimension achievable by this technique. Therefore, during the modeling procedure a fixed value according to SEM images (i.e. 5 μm) is used. This value is not critical and its modification does not affect the fitting result.

II. Shell radius values obtained as a fitting result have a physical meaning because they are generally in the nm range.

III. Polydispersity is the parameter describing the shell dimensional distribution. Lower polydispersity values correspond to more evident bumps in the profile and this is linked to more homogeneous wall dimensions.

Scattering length densities describe how strong is the interaction between X-rays and the different phases of the investigated system. In the case of core-shell model it is necessary to consider three different scattering length density values: that for the core, one for shell and one for dispersing medium. It is possible to calculate these values by exactly knowing the chemical formulas, compositions and densities of all the phases. In the present case these values have been fixed according to experimental conditions.

### Bump Descriptor Value Calculation

For a model-independent quantification of the capsules effectiveness a new parameter was defined, the so-called “bump descriptor” (BD). BD is calculated according to equation 3 from the difference between the experimental curve and an ideal power law curve interpolating the experimental points:

$$B D = \frac{1}{N} {\sum }_{i} {( \frac{{I}_{i} - {P}_{i}}{{σ}_{i}} )}^{2} \qquad Equation 3$$

where N is the number of considered points (covering the region where the bumps occur), Iᵢ is the intensity of the experimental points, Pᵢ is the ideal power law curve and σᵢ is the error of the experimental values.

Pᵢ=bkg+AQᵢ^{-B} Equation 4

where bkg is a constant describing the high-Q behavior, A is an amplitude and B is the power law exponent.

It is worthwhile to note that the BD value is strictly related to the Q-range considered for the calculation and to the instrument used. The Q-range here analyzed is 0.009-0.048 Å⁻¹. A standard deviation of ±1 is determined for the BD parameter.

Viscosity Test Method—The viscosity of fluid detergents herein, namely Vₙ, and V_{d}, is measured using a TA AR550 Rheometer, manufactured by TA Instruments Ltd.

Bilton Center, Cleeve Road Letherhead Surrey KT22 7UQ, United Kingdom.

The software used is provided with the instrument and called “Rheology Advantage Instrument Control AR”.

The instrument is set up before each measurement according to the instructions reported in the Manual “AR550 Rheometer Instrument and accessory manual” (January 2004, PN500034.001 rev F) p 25-29, 40-44, and the Manual “Rheology advantage Instrument Control Getting Started Guide” (January 2004, Revision E) p 9-14, 20, 25-28, 37-38. The settings and parameters used are described herein.

In the “Geometry” section of the software (see Rheology advantage Instrument Control Getting Started Guide” (January 2004, Revision E) p 9), the gap between the rotating plate (40 mm steel plate) and the sample platform (Peltier plate) is set at 500 microns. The procedure is a continuous ramp test, i.e. a procedure in which the rheology of the sample is measured versus increasing shear rate. The setting for the shear rate ranges from 0.04 s⁻¹ to 30 s⁻¹ with a total duration of 3 minutes for the continuous ramp test, and sampling of 20 points per each tenfold increase in shear rate (automatically done), providing in total 60 measurements. Temperature is set at 21° C.

A sample of compact fluid laundry detergent composition according to the invention, or a sample of a non-inventive laundry detergent for purposes of comparison is loaded into the rehometer using a loading procedure as described herein. The sample loading procedure (as described in detail in the manual) is as follows:
1. The temperature is checked (see “instrument status” section) to see if it matches the set temperature. If the temperature is not correct, the settings need to be verified following the instructions in the manual.
2. The sample is loaded using a plastic pipette with a minimum diameter of 4 mm at the tip (to minimize the impact of the stress carried out by the loading action on the rheology of the sample). A minimum amount of 5 ml needs to be applied in the center of the peltier plate to assure full product coverage of the rotating plate.
3. The rotating plate (plate connected to the measuring system) is brought to the set distance (as defined above).
4. The excess of sample (i.e. any sample that may be around the edges of the rotating plate) is removed with a spatula assuring correct loading of the sample according to the description in the manual.
   The measurement steps are as follows:
5. After the sample is loaded, it needs to be left for 10 seconds at rest. The run is started, while making sure the equipment is not exposed to vibrations during the measurement, as this will effect the results. In the case that the measurement is influenced by vibrations, the experiment is repeated whilst excluding the source of vibration.
6. At the end of the run the program stops automatically. All viscosity data are automatically saved.
7. The plates are cleaned with water and ethanol and then dried with paper towel.

The viscosity data, Vn, quoted herein is determined at a shear rate of 20 s-1

The data quoted in the patent examples refer to a shear rate of 20 s-1. In case no measurement was taken at exactly 20 s-1, the data are calculated based on interpolation of the data points which are closest to the 20 s-1 point.

### Fracture Strength Test Method

a.) Place 1 gram of particles in 1 liter of distilled deionized (DI) water.
b.) Permit the particles to remain in the DI water for 10 minutes and then recover the particles by filtration, using a 60 mL syringe filter, 1.2 micron nitrocellulose filter (Millipore, 25 mm diameter).
c.) Determine the rupture force of 50 individual particles. The rupture force of a particle is determined using the procedure given in Zhang, Z.; Sun, G; “Mechanical Properties of Melamine-Formaldehyde microcapsules,” J. Microencapsulation, vol 18, no. 5, pages 593-602, 2001. Then calculate the fracture strength of each particle by dividing the rupture force (in Newtons) by the cross-sectional area of the respective spherical particle (πr², where r is the radius of the particle before compression), said cross-sectional area being determined as follows: measuring the particle size of each individual particle using the experimental apparatus and method of Zhang, Z.; Sun, G; “Mechanical Properties of Melamine-Formaldehyde microcapsules,” J. Microencapsulation, vol 18, no. 5, pages 593-602, 2001.
d.) Use the 50 independent measurements from c.) above, and calculate the percentage of particles having a fracture strength within the claimed range fracture strength range.

### C log P Test

The “calculated log P” (C log P) is determined by the fragment approach of Hansch and Leo (cf., A. Leo, in Comprehensive Medicinal Chemistry, Vol. 4, C. Hansch, P. G. Sammens, J. B. Taylor, and C. A. Ramsden, Eds. P. 295, Pergamon Press, 1990, incorporated herein by reference). C log P values may be calculated by using the “C LOG P” program available from Daylight Chemical Information Systems Inc. of Irvine, Calif. U.S.A.

### Boiling Point Test

Boiling point is measured by ASTM method D2887-04a, “Standard Test Method for Boiling Range Distribution of Petroleum Fractions by Gas Chromatography,” ASTM International.

### Odor Detection Threshold (ODT)

Odour detection threshold is determined using the protocol found in U.S. Pat. No. 6,869,923 B1, from Column 3, line 39 through Column 4, line 15.

### Particle Size Test

a) Place 1 gram of particles in 1 liter of distilled deionized (DI) water.
b) Permit the particles to remain in the DI water for 10 minutes and then recover the particles by filtration, using a 60 mL syringe filter, 1.2 micron nitrocellulose filter (Millipore, 25 mm diameter).
c) Determine the particle size of 50 individual particles using the experimental apparatus and method of Zhang, Z.; Sun, G; “Mechanical Properties of Melamine-Formaldehyde microcapsules,” J. Microencapsulation, vol 18, no. 5, pages 593-602, 2001.
d) Use the 50 independent measurements from c.) above, and calculate the percentage of particles having a particle size within the claimed range.

### Particle Wall Thickness Test

All references to Leica Microsystems refer to the Company with Corporate Headquarters located at:

### Leica Microsystems GmbH

### Ernst-Leitz-Strasse 17-37

### 35578 Wetzlar

All references to Drummond refer to the Company located at:

### Drummond Scientific Company

### 500 Parkway, Box 700

### Broomall, Pa. 19008

All references to Hitachi refer to the Company with Corporate Headquarters located at:

### Hitachi High Technologies

24-14, Nishi-Shimbashi 1-chome, Minato-ku,

### Tokyo 105-8717, Japan

All references to Gatan refer to the Company with Corporate Headquarters located at:

### Gatan, Inc.

### 5933 Coronado Lane

### Pleasanton, Calif. 94588

All references to Quartz refer to the Company with offices located at:

### Quartz Imaging Corporation

### Technology Enterprise Facility III

### 6190 Agronomy Rd, Suite 406

### Vancouver, B.C. Canada V6T 1Z3

### Materials:

Methylcyclohexane—Alfa Aesar Catalogue Number A16057 or equivalent
Capillary Pipettes—Drummond Catalogue Number 5-000-1005 or equivalent
Flat Specimen Carrier—Leica Microsystems P/N 706897 or equivalent
Copper Washers—Leica Microsystems P/N 706867 or equivalent
Flat Specimen Pod—Leica Microsystems P/N 706839 or equivalent
Loading Device for Flat Specimen Holder—Leica Microsystems P/N 706832 or equivalent
Torque Wrench—Leica Microsystems P/N 870071 or equivalent
Allen Bit, 2 mm—Leica Microsystems P/N 870072 or equivalent
Forceps—Leica Microsystems P/N 840105 or equivalent

### Gatan Planchette Collet—Gatan P/N PEP5099

### Gatan Planchette Specimen Holder—Gatan P/N PEP1395

### Instruments:

Scanning Electron Microscope—Hitachi Model S-5200 SEM/STEM or equivalent
High Pressure Freezer—Leica Microsystems Model 706802 EM Pact or equivalent
Cryotransfer Device—Gatan Model CT3500 or equivalent
Cryotransfer System—Gatan Model CT2500 or equivalent
Gatan ITC Temperature Controller—Gatan Model ITC502 or equivalent
Image Analysis Software—Quartz PCI Version 5 or equivalent
Sample: Obtain the sample of microcapsules as per the procedure of 1 above entitled “Fracture Strength”. 50 samples are required.

### Test Procedure

1) Turn on the Leica Microsystems High Pressure Freezer (Leica Microsystems Model Number 706802).
2) Fill up the methylcyclohexane container on the High Pressure Freezer with methylcyclohexane (Alfa Aesar Cat. # A16057 or equivalent).
3) Fill up the liquid nitrogen dewar on the High Pressure Freezer.
4) Fill the liquid nitrogen bath on the High Pressure Freezer
5) The display on the High Pressure Freezer will show Load Sample on the front panel when the instrument is ready to use.
6) Start the Hitachi Model S-5200 SEM/STEM and set the Accelerating Voltage to 3.0 KV and the Emission Current to 20 μA.
7) Fill the Anti-contaminator Dewar located on the lower right side of the Hitachi Model S-5200 SEM/STEM microscope column with liquid nitrogen.
8) Fill the liquid nitrogen dewar on the Gatan Alto 2500 Cryotransfer System (Gatan Model CT2500). Replenish the liquid nitrogen until the dewar remains full. The device is ready to use when the prepchamber temperature reads below −190° C.
9) Place a copper washer (Leica Microsystems P/N 706867) on top of the flat specimen carrier such that the hole in the washer aligns with the well in the flat specimen carrier.
10) Take a glass capillary pipette (Drummond P/N 5-000-1005 or similar) and insert the provided wire plunger into one end of the pipette
11) Insert the pipette into the microcapsule dispersion and withdraw the plunger part way to pull a few microliters of the dispersion into the pipette.
12) Place the tip of the pipette in the well in the flat specimen carrier and push the plunger into the pipette to dispense a small amount of liquid until the well is just slightly overfilled.
13) Insert a 2 mm Allen key bit (Leica Microsystems P/N 870072) into the torque wrench (Leica Microsystems P/N 870071).
14) Using the torque wrench with the bit, loosen the Diamond Locking Screw in the Flat Specimen Pod (Leica Microsystems P/N 706839).
15) Place the Flat Specimen Holder and Copper Washer into the Flat Specimen Pod.
16) Use the torque wrench with the 2 mm Allen key bit to tighten the Diamond Locking Screw in the Flat Specimen Pod onto the specimen until the torque wrench clicks twice.
17) Attach the Loading Device for the Flat Specimen Holder (Leica Microsystems P/N 706832) to the Flat Specimen Pod by screwing it onto the exposed threads of the Diamond Locking Screw.
18) Place the Loading Device for the Flat Specimen Holder with the Flat Specimen Pod onto the EM Pact High Pressure Freezer (Leica Microsystems P/N 706802) and insert it into the High Pressure Freezer.
19) Freeze the specimen using the High Pressure Freezer.
20) Transfer the Flat Specimen Pod to the Unloading Station and unscrew the Loading Device for the Flat Specimen Carrier being careful to keep it immersed in the liquid nitrogen bath.
21) Using the torque wrench, loosen the Diamond Locking Screw.
22) Using tweezers with the tips cooled in liquid nitrogen until the liquid nitrogen stops boiling, remove the Flat Specimen Carrier from the Flat Specimen Pod and place it into a small container in the liquid nitrogen bath.
23) Place the Gatan CT3500 Cryotransfer Device (Gatan Model Number CT3500) into the Gatan Specimen Workstation.
24) Fill the liquid nitrogen dewar on the Gatan CT3500 Cryotransfer device and fill the dewar on the Gatan Specimen Workstation replenishing the liquid nitrogen as necessary until rapid boiling of the liquid nitrogen stops.
25) Transfer the Flat Specimen Holder to the Gatan Specimen Workstation while keeping it in a container of liquid nitrogen.
26) Using tweezers cooled in liquid nitrogen until the liquid nitrogen stops boiling, place the flat specimen holder into the Gatan Planchette Collet (Gatan P/N PEP5099) and press down firmly.
27) Place the assembly from step 26 into the Gatan Planchette Specimen Holder (Gatan P/N PEP1395) and press down firmly.
28) Push the Gatan Cryotransfer device back into the Gatan Specimen Workstation.
29) Using the Gatan supplied 5 mm Friction Tool, screw the Gatan Planchette Specimen Holder into the Gatan Cryotransfer device.
30) Remove the Gatan Cryotransfer device from the Gatan Specimen Workstation and insert it into the Gatan Alto 2500 Cryotransfer System.
31) Attach the Gatan ITC Temperature Controller (Gatan Model Number ITC502) to the Gatan Cryotransfer device by attaching the Temperature Measurement Lead from the Gatan ITC controller to the connector on top of the Gatan Cryotransfer device.
32) Using the Gatan ITC Controller, raise the temperature of the specimen to −120° C.
33) Using the fracturing knife, break off the copper washer to fracture the specimen.
34) Reduce the temperature of the specimen below −160° C.
35) With the voltage set to 6 KV and the gas flow set to provide 10 mA sputter current, press the sputter button and once the current displays 10 mA, let the coater run for 60-90 seconds coating the specimen with gold/palladium.
36) Close the frost shield on the Gatan CT3500 Cryotransfer Device and transfer the specimen to the Hitachi S-5200 SEM/STEM.
37) Wait for the temperature of the Gatan CT3500 Cryotransfer device to stabilize, typically between −170° C. and −172° C.
38) Open the frost shield on the Gatan CT3500 Cryotransfer device by turning the frost shield control knob counter-clockwise.
39) Move the sample around using the stage control trackball, locate a broken microcapsule and adjust the magnification to 50,000 to 150,000×.
40) Adjust the focus and stigmation controls to obtain the best image.
41) Acquire an image of the cross-section of the capsule wall.

### Calculations

1) Select the ruler tool in the Quartz PCI software.
2) Move the cursor to one edge of the microcapsule wall.
3) Click and hold the left mouse button while dragging the mouse cursor to the opposite side of the capsule wall keeping the drawn line perpendicular to the face of the capsule wall to measure the wall thickness.
4) Use 50 independent measurements (1 measurement for each capsule) to calculate the percentage of particles having a wall thickness in the claimed range.

### Benefit Agent Leakage Test

a.) Obtain 2, one gram samples of benefit agent particle composition.
b.) Add 1 gram (Sample 1) of particle composition to 99 grams of product matrix that the particle will be employed in and with the second sample immediately proceed to Step d below.
c.) Age the particle containing product matrix (Sample 1) of a.) above for 2 weeks at 35° C. in a sealed, glass jar.
d.) Recover the particle composition's particles from the product matrix of c.) (Sample 1 in product matrix) and from particle composition (Sample 2) above by filtration.
e.) Treat each particle sample from d.) above with a solvent that will extract all the benefit agent from each samples' particles.
f.) Inject the benefit agent containing solvent from each sample from e.) above into a Gas Chromatograph and integrate the peak areas to determine the total quantity of benefit agent extracted from each sample.
g.) The benefit agent leakage is defined as:
   Value from f.) above for Sample 2−Value from f.) above for Sample 1.

### EXAMPLES

### Example 1

### Melamine Formaldehyde (MF) Capsule

A first solution is created after 70 grams of water, 7 grams of butyl acrylate-acrylic acid copolymer emulsifier (Colloid C351, 25% solids, pka 4.5-4.7, (Kemira Chemicals, Inc. Kennesaw, Ga. U.S.A.) and 4.5 grams of polyacrylic acid (35% solids, pka 1.5-2.5, Aldrich) are charged into a vessel and mixed until homogeneous and heated to 60 C. The pH of the solution is adjusted to 6.0 with sodium hydroxide solution. 12.7 grams of water and 4.2 grams of partially methylated methylol melamine resin (Cymel 385, 80% solids, (Cytec Industries West Paterson, N.J., U.S.A.)) are added to the solution. 70 grams of perfume oil is added to the previous liquor under mechanical agitation. The resulting mixture is emulsified under high shear agitation.

A second solution consisting of 42 grams of water, 3 grams of polyacrylic acid (35% solids, pka 1.5-2.5, Aldrich) is adjusted to a pH of 5.1 with sodium hydroxide. 12 grams of partially methylated methylol melamine resin (Cymel 385, 80% solids, (Cytec Industries West Paterson, N.J., U.S.A.)) and 9 grams of water are added to the solution. This second solution is then added to the first composition.

### Example 2

### Melamine Formaldehyde (MF) Capsule

A first solution is created after 63.3 grams of water, 6.6 grams of butyl acrylate-acrylic acid copolymer emulsifier (Colloid C351, 25% solids, pka 4.5-4.7, (Kemira Chemicals, Inc. Kennesaw, Ga. U.S.A.) and 4.7 grams of polyacrylic acid (35% solids, pka 1.5-2.5, Aldrich) are charged into a vessel and mixed until homogeneous and heated to 65 C. The pH of the solution is adjusted to 5.8 with sodium hydroxide solution. 12.7 grams of water and 2.8 grams of partially methylated methylol melamine resin (Cymel 385, 80% solids, (Cytec Industries West Paterson, N.J., U.S.A.)) are added to the solution. 75.3 grams of perfume oil is added to the previous liquor under mechanical agitation. The resulting mixture is emulsified under high shear agitation.

A second solution consisting of 36.1 grams of water, 1.5 grams of polyacrylic acid (35% solids, pka 1.5-2.5, Aldrich) is adjusted to a pH of 4.95 with sodium hydroxide. 4.5 grams of partially methylated methylol melamine resin (Cymel 385, 80% solids, (Cytec Industries West Paterson, N.J., U.S.A.)) and 9 grams of water are added to the solution. This second solution is then added to the first composition.

1.8 grams of sodium sulfate salt are added to the emulsion under agitation. This mixture is heated to 85 degree. C and then maintained overnight with continuous stirring to complete the encapsulation process. 8 grams of acetoacetamide (Sigma-Aldrich, Saint Louis, Mo., U.S.A.) is added to the suspension. An average capsule size of 20 um is obtained as analyzed by a Model 780 Accusizer.

### Example 3

17 grams of butyl acrylate-acrylic acid copolymer emulsifier (Colloid C351, 25% solids, pka 4.5-4.7, (Kemira Chemicals, Inc. Kennesaw, Ga. U.S.A.) and 17 grams of polyacrylic acid (35% solids, pKa 1.5-2.5, Aldrich) are dissolved and mixed in 200 grams deionized water. The pH of the solution is adjusted to pH of 6.0 with sodium hydroxide solution. 7 grams of partially methylated methylol melamine resin (Cymel 385, 80% solids, (Cytec Industries West Paterson, N.J., U.S.A.)) is added to the emulsifier solution. 200 grams of perfume oil is added to the previous mixture under mechanical agitation and the temperature is raised to 45° C. After mixing at higher speed until a stable emulsion is obtained, the second solution and 4 grams of sodium sulfate salt are added to the emulsion. This second solution contains 3 grams of polyacrylic acid polymer (Colloid C121, 25% solids (Kemira Chemicals, Inc. Kennesaw, Ga. U.S.A.), 100 grams of distilled water, sodium hydroxide solution to adjust pH to 6.0, 10 grams of partially methylated methyol melamine resin (Cymel 385, 80% Cytec). This mixture is heated till 85 C and maintained 8 hours with continuous stifling to complete the encapsulation process. 23 grams of acetoacetamide (Sigma-Aldrich, Saint Louis, Mo. U.S.A.) is added to the suspension. Salts and structuring agents can then still be added to the slurry.

### Example 4

### Melamine Formaldehyde Capsule

The composition of and the procedures for preparing the capsules are the same composition as in Example 2 except for the following: 0.7% of ammonium hydroxide is added to the suspension instead of acetoacetamide.

### Example 5

### Production of Spray Dried Microcapsule

1200 g of perfume microcapsule slurry, containing one or more of the variants of microcapsules disclosed in the present specification, is mixed together with 700 g of water for 10 minutes using an IKA Eurostar mixer with R1382 attachment at a speed of 180 rpm. The mixture is then transferred over to a feeding vessel to be spray dried in a 1.2 m diameter Niro Production Minor. The slurry is fed into the tower using a Watson-Marlow 504U peristaltic pump and atomised using a 100 mm diameter rotary atomiser run at 18000 rpm, with co-current air flow for drying. The slurry is dried using an inlet temperature of 200° C. and outlet temperature of 95° C. to form a fine powder. The equipment used the spray drying process may be obtained from the following suppliers: IKA Werke GmbH & Co. KG, Janke and Kunkel—Str. 10, D79219 Staufen, Germany; Niro A/S Gladsaxevej 305, P.O. Box 45, 2860 Soeborg, Denmark and Watson-Marlow Bredel Pumps Limited, Falmouth, Cornwall, TR11 4RU, England.

### Example 6

1.28 kg of precipitated silica Sipernat® 22S (Degussa) is added to an F-20 paddle mixer (Forberg). The mixer is run initially for 5 seconds to distribute the silica evenly on the base of the mixer. The mixer is stopped and 8.25 kg of paste, made according to Example 2, is evenly distributed onto the powder. The mixer is then run at 120 rpm for a total of 30 seconds.

Following mixing, the wet particles are dumped out of the mixer and screened using a 2000 micron sieve to remove the oversize. The product passing through the screen is dried in 500 g batches in a CDT 0.02 fluid bed dryer (Niro) to a final moisture content of 20 wt % measured by Karl Fischer. The dryer is operated at an inlet temperature of 140° C. and air velocity of 0.68 m/s.

### Examples 7-14

Examples of laundry detergent compositions comprising the perfume composition are included below.

| | % w/w of laundry detergent compositions | | | | | | | |
|---|---|---|---|---|---|---|---|---|
| Raw material | 7 | 8 | 9 | 10 | 11 | 12 | 13 | 14 |
| Linear alkyl benzene | 7.1 | 6.7 | 11.0 | 10.6 | 6.9 | 4.5 | 10.1 | 8.9 |
| sulphonate | | | | | | | | |
| Sodium C₁₂₋₁₅ alkyl ethoxy | 3.5 | 0.0 | 1.5 | 0.0 | 0.0 | 0.0 | 0.0 | 1.9 |
| sulphate having a molar | | | | | | | | |
| average degree of | | | | | | | | |
| ethoxylation of 3 | | | | | | | | |
| Acrylic Acid/Maleic Acid | 3.6 | 1.8 | 4.9 | 2.0 | 1.0 | 1.6 | 3.9 | 2.3 |
| Copolymer | | | | | | | | |
| Sodium Alumino Silicate | 4.0 | 0.5 | 0.8 | 1.4 | 16.3 | 0.0 | 17.9 | 2.4 |
| (Zeolite 4A) | | | | | | | | |
| Sodium Tripolyphosphate | 0.0 | 17.5 | 0.0 | 15.8 | 0.0 | 23.3 | 0.0 | 0.0 |
| Sodium Carbonate | 23.2 | 16.8 | 30.2 | 17.3 | 18.4 | 9.0 | 20.8 | 30.0 |
| Sodium Sulphate | 31.4 | 29.4 | 35.5 | 7.2 | 26.3 | 42.8 | 33.2 | 28.3 |
| Sodium Silicate | 0.0 | 4.4 | 0.0 | 4.5 | 0.0 | 6.1 | 0.0 | 4.6 |
| C₁₄₋₁₅ alkyl ethoxylated | 0.4 | 2.6 | 0.8 | 2.5 | 3.1 | 0.3 | 3.8 | 0.4 |
| alcohol having a molar | | | | | | | | |
| average degree of | | | | | | | | |
| ethoxylation of 7 | | | | | | | | |
| Sodium Percarbonate | 16.0 | 0.0 | 8.4 | 20.4 | 13.1 | 3.6 | 0.0 | 7.0 |
| Sodium Perborate | 0.0 | 9.9 | 0.0 | 0.0 | 0.0 | 0.0 | 0.0 | 0.0 |
| Tetraacetylethylenediamine | 2.2 | 1.7 | 0.0 | 4.7 | 3.6 | 0.0 | 0.0 | 0.8 |
| (TAED) | | | | | | | | |
| Calcium Bentonite | 0.0 | 0.0 | 0.0 | 1.8 | 0.0 | 0.0 | 0.0 | 5.6 |
| Citric acid | 2.0 | 1.5 | 2.0 | 2.0 | 2.5 | 1.0 | 2.5 | 1.0 |
| Protease (84 mg active/g) | 0.14 | 0.12 | 0.0 | 0.12 | 0.09 | 0.08 | 0.10 | 0.08 |
| Amylase (22 mg active/g) | 0.10 | 0.11 | 0.0 | 0.10 | 0.10 | 0.0 | 0.14 | 0.08 |
| Lipase (11 mg active/g) | 0.70 | 0.50 | 0.0 | 0.70 | 0.50 | 0.0 | 0.0 | 0.0 |
| Cellulase (2.3 mg active/g) | 0.0 | 0.0 | 0.0 | 0.0 | 0.0 | 0.0 | 0.18 | 0.0 |
| Benefit agent composition | 1.4 | — | — | 1.0 | 0.7 | — | — | 1.2 |
| of Example 4 | | | | | | | | |
| Benefit agent composition | — | 0.8 | 1.4 | — | — | 0.5 | 0.7 | — |
| of Example 5 | | | | | | | | |

| | |
|---|---|
| Water & Miscellaneous | Balance to 100% |

### Examples 15-22

Examples of granular laundry detergent compositions comprising the perfume composition are included below.

| | % w/w of laundry detergent compositions | | | | | | | |
|---|---|---|---|---|---|---|---|---|
| Raw material | 15 | 16 | 17 | 18 | 19 | 20 | 21 | 22 |
| Linear alkyl benzene | 7.1 | 6.7 | 11.0 | 10.6 | 6.9 | 4.5 | 10.1 | 8.9 |
| sulphonate | | | | | | | | |
| Sodium C₁₂₋₁₅ alkyl ethoxy | 3.5 | 0.0 | 1.5 | 0.0 | 0.0 | 0.0 | 0.0 | 1.9 |
| sulphate having a molar | | | | | | | | |
| average degree of | | | | | | | | |
| ethoxylation of 3 | | | | | | | | |
| Acrylic Acid/Maleic Acid | 3.6 | 1.8 | 4.9 | 2.0 | 1.0 | 1.6 | 3.9 | 2.3 |
| Copolymer | | | | | | | | |
| Sodium Alumino Silicate | 4.0 | 0.5 | 0.8 | 1.4 | 16.3 | 0.0 | 17.9 | 2.4 |
| (Zeolite 4A) | | | | | | | | |
| Sodium Tripolyphosphate | 0.0 | 17.5 | 0.0 | 15.8 | 0.0 | 23.3 | 0.0 | 0.0 |
| Sodium Carbonate | 23.2 | 16.8 | 30.2 | 17.3 | 18.4 | 9.0 | 20.8 | 30.0 |
| Sodium Sulphate | 31.4 | 29.4 | 35.5 | 7.2 | 26.3 | 42.8 | 33.2 | 28.3 |
| Sodium Silicate | 0.0 | 4.4 | 0.0 | 4.5 | 0.0 | 6.1 | 0.0 | 4.6 |
| C₁₄₋₁₅ alkyl ethoxylated | 0.4 | 2.6 | 0.8 | 2.5 | 3.1 | 0.3 | 3.8 | 0.4 |
| alcohol having a molar | | | | | | | | |
| average degree of | | | | | | | | |
| ethoxylation of 7 | | | | | | | | |
| Sodium Percarbonate | 16.0 | 0.0 | 8.4 | 20.4 | 13.1 | 3.6 | 0.0 | 7.0 |
| Sodium Perborate | 0.0 | 9.9 | 0.0 | 0.0 | 0.0 | 0.0 | 0.0 | 0.0 |
| Tetraacetylethylenediamine | 2.2 | 1.7 | 0.0 | 4.7 | 3.6 | 0.0 | 0.0 | 0.8 |
| (TAED) | | | | | | | | |
| Calcium Bentonite | 0.0 | 0.0 | 0.0 | 1.8 | 0.0 | 0.0 | 0.0 | 5.6 |
| Citric acid | 2.0 | 1.5 | 2.0 | 2.0 | 2.5 | 1.0 | 2.5 | 1.0 |
| Protease (84 mg active/g) | 0.14 | 0.12 | 0.0 | 0.12 | 0.09 | 0.08 | 0.10 | 0.08 |
| Amylase (22 mg active/g) | 0.10 | 0.11 | 0.0 | 0.10 | 0.10 | 0.0 | 0.14 | 0.08 |
| Lipase (11 mg active/g) | 0.70 | 0.50 | 0.0 | 0.70 | 0.50 | 0.0 | 0.0 | 0.0 |
| Cellulase (2.3 mg active/g) | 0.0 | 0.0 | 0.0 | 0.0 | 0.0 | 0.0 | 0.18 | 0.0 |
| Benefit agent composition | 1.4 | 0.6 | 0.8 | 1.0 | 0.7 | 0.3 | 0.7 | 1.2 |
| of Example 5 | | | | | | | | |

| | |
|---|---|
| Water & Miscellaneous | Balance to 100% |

The equipment and materials described in Examples 6 through to 21 can be obtained from the following: IKA Werke GmbH & Co. KG, Staufen, Germany; CP Kelco, Atlanta, United States; Forberg International AS, Larvik, Norway; Degussa GmbH, Düsseldorf, Germany; Niro A/S, Soeberg, Denmark; Baker Perkins Ltd, Peterborough, United Kingdom; Nippon Shokubai, Tokyo, Japan; BASF, Ludwigshafen, Germany; Braun, Kronberg, Germany; Industrial Chemicals Limited, Thurrock, United Kingdom; Primex ehf, Siglufjordur, Iceland; ISP World Headquarters; Polysciences, Inc. of Warrington, Pa., United States; Cytec Industries Inc., New Jersey, United States; International Specialty Products, Wayne, N.J., United States; P&G Chemicals Americas, Cincinnati, Ohio, United States; Sigma-Aldrich Corp., St. Louis, Mo., United States, Dow Chemical Company of Midland, Mich., USA

### Examples 23-32

### Fabric Conditioner

Non-limiting examples of fabric conditioners containing the polymer coated perfume microcapsules disclosed in the present specification are summarized in the following table.

| | EXAMPLES | | | | | | | | | |
|---|---|---|---|---|---|---|---|---|---|---|
| (% wt) | 23 | 24 | 25 | 26 | 27 | 28 | 29 | 30 | 31 | 32 |
| FSA ^{a} | 14 | 16.47 | 14 | 12 | 12 | 16.47 | — | — | 5 | 10 |
| FSA ^{b} | | | | | — | | 3.00 | — | — | — |
| FSA ^{c} | | | | | — | | — | 6.5 | — | — |
| Ethanol | 2.18 | 2.57 | 2.18 | 1.95 | 1.95 | 2.57 | — | — | 0.81 | |
| Isopropyl | — | — | — | — | — | — | 0.33 | 1.22 | — | 1.0— |
| Alcohol | | | | | | | | | | |
| Starch ^{d} | 1.25 | 1.47 | 2.00 | 1.25 | — | 2.30 | 0.5 | 0.70 | 0.71 | — |
| Phase | 0.21 | 0.25 | 0.21 | 0.21 | 0.14 | 0.18 | 0.15 | 0.14 | 0.2 | 0.15 |
| Stabilizing | | | | | | | | | | |
| Polymer ^{f} | | | | | | | | | | |
| Suds | — | — | — | — | — | — | — | 0.1 | — | — |
| Suppressor ^{g} | | | | | | | | | | |
| Calcium | 0.15 | 0.176 | 0.15 | 0.15 | 0.30 | 0.176 | — | 0.1-0.15 | — | 0.025 |
| Chloride | | | | | | | | | | |
| DTPA ^{h} | 0.017 | 0.017 | 0.017 | 0.017 | 0.007 | 0.007 | 0.20 | — | 0.002 | — |
| Preservative | 5 | 5 | 5 | 5 | 5 | 5 | — | 250 ^{j} | 5 | 5 |
| (ppm) ^{i, j, l} | | | | | | | | | | |
| Antifoam ^{k} | 0.015 | 0.018 | 0.015 | 0.015 | 0.015 | 0.015 | — | — | 0.015 | 0.015 |
| Dye | 40 | 40 | 40 | 40 | 40 | 40 | 11 | 30-300 | 30 | 30 |
| (ppm) | | | | | | | | | | |
| Ammonium | 0.100 | 0.118 | 0.100 | 0.100 | 0.115 | 0.115 | — | — | — | — |
| Chloride | | | | | | | | | | |
| HCl | 0.012 | 0.014 | 0.012 | 0.012 | 0.028 | 0.028 | 0.016 | 0.025 | 0.011 | 0.011 |
| Perfume | 0.2 | 0.02 | 0.1 | 0.15 | 0.12 | 0.13 | 0.3 | 0.4 | 0.24 | 0.23 |
| microcapsules as | | | | | | | | | | |
| disclosed in | | | | | | | | | | |
| Example 1 | | | | | | | | | | |
| Additional | 0.8 | 0.7 | 0.9 | 0.5 | 1.2 | 0.5 | 1.1 | 0.6 | 1.0 | 0.9 |
| Neat | | | | | | | | | | |
| Perfume | | | | | | | | | | |
| Deionized | † | † | † | † | † | † | † | † | † | † |
| Water | | | | | | | | | | |

^{a} N,N-di(tallowoyloxyethyl)-N,N-dimethylammonium chloride.
^{b} Methyl bis(tallow amidoethyl)2-hydroxyethyl ammonium methyl sulfate.
^{c} Reaction product of Fatty acid with Methyldiethanolamine in a molar ratio 1.5:1, quaternized with Methylchloride, resulting in a 1:1 molar mixture of N,N-bis(stearoyl-oxy-ethyl) N,N-dimethyl ammonium chloride and N-(stearoyl-oxy-ethyl) N,-hydroxyethyl N,N dimethyl ammonium chloride.
^{d} Cationic high amylose maize starch available from National Starch under the trade name CATO ®.
^{f} Rheovis DCE ex BASF.
^{g} SE39 from Wacker
^{h} Diethylenetriaminepentaacetic acid.
ⁱ KATHON ® CG available from Rohm and Haas Co. “PPM” is “parts per million.”
^{j} Gluteraldehyde
^{l} Proxel GXL
^{k} Silicone antifoam agent available from Dow Corning Corp. under the trade name DC2310.
† balance

### Examples 33-35

### Liquid and Gel Detergents

TABLE 1
(% by Weight)

| Ingredients | 33 | 34 | 35 |
|---|---|---|---|
| Alkylbenzenesulfonic acid | 17.2 | 12.2 | 23 |
| C12-14 alcohol 7-ethoxylate | 8.6 | 0.4 | 19.5 |
| C14-15 alcohol 8-ethoxylate | — | 9.6 | — |
| C12-14 alcohol 3-ethoxylate sulphate, Na | 8.6 | — | — |
| salt | | | |
| C8-10 Alkylamidopropyldimethyl amine | — | — | 0.9 |
| Citric acid | 2.9 | 4.0 | — |
| C12-18 fatty acid | 12.7 | 4.0 | 17.3 |
| Enzymes | 3.5 | 1.1 | 1.4 |
| Ethoxylated polyimine | 1.4 | — | 1.6 |
| Ethoxylated polyimine polymer, quaternized | 3.7 | 1.8 | 1.6 |
| and sulphated | | | |
| Hydroxyethane diphosphonic acids (HEDP) | 1.4 | — | — |
| Pentamethylene triamine pentaphosphonic | — | 0.3 | — |
| acid | | | |
| Catechol 2,5 disulfonate, Na salt | 0.9 | — | — |
| Fluorescent whitening agent | 0.3 | 0.15 | 0.3 |
| 1,2 propandiol | 3.5 | 3.3 | 22 |
| Ethanol | — | 1.4 | — |
| Diethylene glycol | — | 1.6 | — |
| 1-ethoxypentanol | 0.9 | — | — |
| Sodium cumene sulfonate | | 0.5 | — |
| Monoethanolamine (MEA) | 10.2 | 0.8 | 8.0 |
| MEA borate | 0.5 | 2.4 | — |
| Sodium hydroxide | — | 4.6 | — |
| Perfume | 1.6 | 0.7 | 1.5 |
| Perfume microcapsules as Example 2 | 1.1 | 1.2 | 0.9 |
| Water | 22.1 | 50.8 | 2.9 |
| Perfume, dyes, miscellaneous minors | Balance | Balance | Balance |
| Undiluted viscosity (Vₙ) at 20 s⁻¹, cps | 2700 | 400 | 300 |

### Example 36

### Liquid Unit Dose

The following are examples of unit dose executions wherein the liquid composition is enclosed within a PVA film. The preferred film used in the present examples is Monosol M8630 76 μm thickness.

| | D | | | E | | F | | |
|---|---|---|---|---|---|---|---|---|
| | 3 compartments | | | 2 compartments | | 3 compartments | | |
| | Compartment # | | | | | | | |
| | 42 | 43 | 44 | 45 | 46 | 47 | 48 | 49 |
| | Dosage (g) | | | | | | | |
| | 34.0 | 3.5 | 3.5 | 30.0 | 5.0 | 25.0 | 1.5 | 4.0 |
| Ingredients | Weight % | | | | | | | |
| Alkylbenzene sulfonic | 20.0 | 20.0 | 20.0 | 10.0 | 20.0 | 20.0 | 25 | 30 |
| acid | | | | | | | | |
| Alkyl sulfate | | | | 2.0 | | | | |
| C₁₂₋₁₄ alkyl 7- | 17.0 | 17.0 | 17.0 | | 17.0 | 17.0 | 15 | 10 |
| ethoxylate | | | | | | | | |
| C₁₂₋₁₄ alkyl ethoxy 3 | 7.5 | 7.5 | 7.5 | | | 7.5 | 7.5 | |
| sulfate | | | | | | | | |
| Citric acid | 0.5 | | 2.0 | 1.0 | | | | 2.0 |
| Zeolite A | | | | 10.0 | | | | |
| C₁₂₋₁₈ Fatty acid | 13.0 | 13.0 | 13.0 | | 18.0 | 18.0 | 10 | 15 |
| Sodium citrate | | | | 4.0 | 2.5 | | | |
| enzymes | 0-3 | 0-3 | 0-3 | 0-3 | | 0-3 | 0-3 | 0-3 |
| Sodium Percarbonate | | | | 11.0 | | | | |
| TAED | | | | 4.0 | | | | |
| Polycarboxylate | | | | 1.0 | | | | |
| Ethoxylated | 2.2 | 2.2 | 2.2 | | | | | |
| Polyethylenimine¹ | | | | | | | | |
| Hydroxyethane | 0.6 | 0.6 | 0.6 | 0.5 | | | 2.2 | |
| diphosphonic acid | | | | | | | | |
| Ethylene diamine | | | | | | 0.4 | | |
| tetra(methylene | | | | | | | | |
| phosphonic) acid | | | | | | | | |
| Brightener | 0.2 | 0.2 | 0.2 | 0.3 | | 0.3 | | |
| Perfume | 0.4 | 1.2 | 1.5 | 1.3 | 1.3 | 0.4 | 0.12 | 0.2 |
| Microcapsules as | | | | | | | | |
| Example2 | | | | | | | | |
| Water | 9 | 8.5 | 10 | 5 | 11 | 10 | 10 | 9 |
| CaCl2 | | | | | | | 0.01 | |
| Perfume | 1.7 | 1.7 | | 0.6 | | 1.5 | 0.5 | |
| Minors (antioxidant, | 2.0 | 2.0 | 2.0 | 4.0 | 1.5 | 2.2 | 2.2 | 2.0 |
| sulfite, aesthetics, . . .) | | | | | | | | |

| | |
|---|---|
| Buffers (sodium | To pH 8.0 for liquids |
| carbonate, | To RA > 5.0 for powders |
| monoethanolamine)³ | |
| Solvents (1,2 propanediol, | To 100 p |
| ethanol), Sulfate | |

¹Polyethylenimine (MW = 600) with 20 ethoxylate groups per —NH.
³RA = Reserve Alkalinity (g NaOH/dose)

### Example 37

### Liquid Laundry Detergent

| | Liquid Detergent Compositions | | | |
|---|---|---|---|---|
| | Example 1 | Example 2 | Example 3 | Example 4 |
| Ingredient | (Comparative) % | (Invention) % | (Invention) % | (Invention) % |
| Linear Alkylbenzene sulfonic | 15 | 15 | 12 | 12 |
| acid¹ | | | | |
| C12-14 alkyl ethoxy 3 sulfate | 10 | 10 | 8 | 9 |
| MEA salt | | | | |
| C12-14 alkyl 7-ethoxylate | 10 | 10 | 8 | 8 |
| C14-15 alkyl 8-ethoxylate | — | — | — | — |
| C12-18 Fatty acid | 10 | 10 | 10 | 10 |
| Citric acid | 2 | 2 | 3 | 3 |
| Ethoxysulfated | — | — | — | 2.2 |
| Hexamethylene Diamine | | | | |
| Dimethyl Quat | | | | |
| Soil Suspending Alkoxylated | 3 | 3 | 2.2 | — |
| Polyalkylenimine Polymer² | | | | |
| PEG-PVAc Polymer³ | — | — | 0.9 | 0.9 |
| Hydroxyethane diphosphonic | 1.6 | 1.6 | 1.6 | 1.6 |
| acid | | | | |
| Fluorescent Whitening Agent | 0.2 | 0.2 | 0.2 | 0.2 |
| 1,2 Propanediol | 6.2 | 6.2 | 8.5 | 8.5 |
| Ethanol | 1.5 | 1.5 | — | — |
| Hydrogenated castor oil | 0.75 (introduced | 0.75 (introduced | | |
| derivative structurant | via NaLAS premix) | via MEA LAS premix) | | |
| Boric acid | 0.5 | 0.5 | 0.5 | 0.5 |
| Perfume | 1.7 | 1.7 | 1.7 | 1.7 |
| Perfume microcapsules as | 1.1 | 1.2 | 0.9 | 1.3 |
| Example 2 | | | | |
| Monoethanolamine | To pH 8.0 | | | |
| Protease enzyme | 1.5 | 1.5 | 1.5 | 1.5 |
| Amylase enzyme | 0.1 | 0.1 | 0.1 | 0.1 |
| Mannanase enzyme | 0.1 | 0.1 | 0.1 | 0.1 |
| Cellulase enzyme | — | — | 0.1 | 0.1 |
| Xyloglucanase enzyme | — | — | 0.1 | 0.1 |
| Pectate lyase | — | — | 0.1 | 0.1 |

| | |
|---|---|
| Water and minors (antifoam, | To 100 parts |
| aesthetics, . . .) | |

¹Weight percentage of Linear Alkylbenzene sulfonic acid includes that which added to the composition via the premix
²600 g/mol molecular weight polyethylenimine core with 20 ethoxylate groups per —NH.
³PEG-PVA graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units.

### Example 38

### Shampoo Formulation

| Ingredient | |
|---|---|
| Ammonium Laureth Sulfate (AE₃S) | 6.00 |
| Ammonium Lauryl Sulfate (ALS) | 10.00 |
| Laureth-4 Alcohol | 0.90 |
| Trihydroxystearin ⁽⁷⁾ | 0.10 |
| Perfume microcapsules as disclosed | 0.60 |
| in Example 1 | |
| Sodium Chloride | 0.40 |
| Citric Acid | 0.04 |
| Sodium Citrate | 0.40 |
| Sodium Benzoate | 0.25 |
| Ethylene Diamine Tetra Acetic Acid | 0.10 |
| Dimethicone ^{(9, 10, 11)} | 1.00 ⁽⁹⁾ |
| Water and Minors (QS to 100%) | Balance |

The dimensions and values disclosed herein are not to be understood as being strictly limited to the exact numerical values recited. Instead, unless otherwise specified, each such dimension is intended to mean both the recited value and functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
a) based on total composition weight, from about 0.001% to about 10%, of an encapsulate selected from the group consisting of
(i) an encapsulate comprising a core comprising a benefit agent and a shell that encapsulates said core, said encapsulate's shell comprising cross-linked melamine formaldehyde and having an ATR-FTIR spectrum second derivative 1490:1550 cm⁻¹ (±2 cm⁻¹) peak ratio from about 0.1 to about 0.7;
(ii) an encapsulate comprising a core comprising a benefit agent and a shell that encapsulates said core, said encapsulate's shell comprising cross-linked melamine formaldehyde and having an ATR-FTIR spectrum second derivative 790:813 cm⁻¹ (±2 cm⁻¹) peak ratio from 0 to about 0.1;
(iii) an encapsulate comprising a core comprising a benefit agent and a shell that at least encapsulates said core, said encapsulate's shell having a SAXS Bump Descriptor value from about 2 to about 1,000,000;
(iv) an encapsulate comprising a core comprising a benefit agent and a shell that encapsulates said core, said encapsulate's shell comprising cross-linked melamine formaldehyde and having an ATR-FTIR spectrum second derivative 790:813 cm⁻¹ (±2 cm⁻¹) peak ratio from 0 to about 0.1, and a SAXS Bump Descriptor value from about 2 to about 1,000,000;
(v) mixtures thereof;
said encapsulates having a wall thickness from about 1 nm to about 200 nm; an encapsulate wall thickness polydispersity from about 0.01 to about 0.2; a particle size median from about 1 micron to about 100 microns; and at least 75% of said encapsulates having a fracture strength from about 0.2 MPa to about 10 MPa;
b) a material selected from the group consisting of a surfactant, a builder, a chelating agent, a dye transfer inhibiting agent, a dispersant, an enzyme, an enzyme stabilizer, a catalytic bleaching material, a bleach activator, a polymeric dispersing agent, a clay soil removal/anti-redeposition agent, a brightener, a suds suppressor, a dye, a structure elasticizing agent, a thickener/structurant, a fabric softener, a carrier, a hydrotrope, a pigment, a silicone and mixtures thereof;
said composition being a solid detergent; a liquid detergent comprising, based on total liquid detergent weight, less than about 60% water and having a neat viscosity of from about 10 cps to about 999 cps; a detergent gel comprising, based on total gel weight, less than about 45% water and having a neat viscosity of from about 1,000 cps to about 10,000 cps; a fabric enhancer; a shampoo; a hair conditioner; or a unit dose detergent comprising a detergent and a water soluble film encapsulating said detergent.

2. The composition of claim 1, said composition comprising based on total composition weight, from about 0.001% to about 10% of an encapsulate comprising a core comprising a benefit agent and a shell that encapsulates said core, said encapsulate's shell comprising cross-linked melamine formaldehyde and having an ATR-FTIR second derivative 790:813 cm⁻¹ (±2 cm⁻¹) peak ratio from 0 to about 0.1 and a SAXS Bump Descriptor value from about 2 to about 1,000,000.

3. The composition of claim 1 wherein said encapsulate's shell comprises a material selected from the group consisting of polyethylenes; polyamides; polystyrenes; polyisoprenes; polycarbonates; polyesters; polyacrylates; aminoplasts, in one aspect said aminoplast comprises a polyureas, polyurethane, and/or polyureaurethane, in one aspect said polyurea comprises polyoxymethyleneurea and/or melamine formaldehyde; polyolefins; polysaccharides, in one aspect alginate and/or chitosan; gelatin; shellac; epoxy resins; vinyl polymers; water insoluble inorganics; silicone; and mixtures thereof.

4. The composition of claim 3 wherein said encapsulate's shell comprises melamine formaldehyde and/or cross linked melamine formaldehyde.

5. The composition of claim 1 wherein said encapsulate's benefit agent is selected from the group consisting of a perfume, a cooling agent, a sensate and mixtures thereof.

6. The composition of claim 1 wherein said encapsulate's core comprises perfume.

7. The composition of claim 6 wherein said encapsulate's core comprises, based total core weight, at least 10% of one or more Table 1 perfume raw materials.

8. The composition of claim 4 wherein said encapsulate's core comprises a perfume composition selected from the group consisting of:
a) a perfume composition having a C log P of less than 4.5 to about 2;
b) a perfume composition comprising, based on total perfume composition weight, at least 60% perfume materials having a C log P of less than 4.0 to about 2;
c) a perfume composition comprising, based on total perfume composition weight, at least 35% perfume materials having a C log P of less than 3.5 to about 2;
d) a perfume composition comprising, based on total perfume composition weight, at least 40% perfume materials having a C log P of less than 4.0 to about 2 and at least 1% perfume materials having a C log P of less than 2.0 to about 1;
e) a perfume composition comprising, based on total perfume composition weight, at least 40% perfume materials having a C log P of less than 4.0 to about 2 and at least 15% perfume materials having a C log P of less than 3.0 to about 1.5;
f) a perfume composition comprising, based on total perfume composition weight, at least 1% of a butanoate ester and at least 1% of a pentanoate ester;
g) a perfume composition comprising, based on total perfume composition weight, at least 2% of an ester comprising an allyl moiety and at least 10%, of another perfume comprising an ester moiety;
h) a perfume composition comprising, based on total perfume composition weight, at least 1% of an aldehyde comprising an alkyl chain moiety;
i) a perfume composition comprising, based on total perfume composition weight, at least 2% of a butanoate ester;
j) a perfume composition comprising, based on total perfume composition weight, at least 1% of a pentanoate ester;
k) a perfume composition comprising, based on total perfume composition weight, at least 3% of an ester comprising an allyl moiety and at least 1% of an aldehyde comprising an alkyl chain moiety; and
l) a perfume composition comprising, based on total perfume composition weight, at least 25% of a perfume comprising an ester moiety and at least 1% of an aldehyde comprising an alkyl chain moiety.

9. The composition of claim 1 wherein said composition is a liquid detergent and said encapsulates comprise a density balancing agent is selected from the group consisting of an organic material having a density greater than about 1, an inorganic oxide, inorganic oxy-chloride, inorganic halogenide, a salt, and mixtures thereof.

10. The composition of claim 1 wherein said encapsulates have a core to wall ratio from about 70:30 to about 98:2.

11. A method of cleaning or treating a situs comprising optionally washing and/or rinsing said situs, contacting said situs with the composition selected from the compositions of claims 1-10 and mixtures thereof and optionally washing and/or rinsing said situs.

12. A process of making an encapsulate comprising:
a) preparing a first solution comprising, based on total solution weight from about 20% to about 90%, a first emulsifier and a first resin, the ratio of said first emulsifier and said first resin being from about from 1:10 to about 10:1;
b) preparing a second solution comprising based on total solution weight from about 20% to about 95% water, a second emulsifier and a second resin, the ratio of said second emulsifier and said second resin being from about 1:100 to about 10:1;
c) combining a core material and said first solution to form a first composition;
d) emulsifying said first composition;
e) for the first and second solution the pH is adjusted from about 3 to about 7;
f) for the first solution the temperature of operation is from about 40° C. to about 90° C.;
g) for the second solution the temperature of operation is from about 5° C. to about 50° C.;
h) combining said first composition and said second solution to form a second composition and optionally combining any processing aids and said second composition;
i) mixing said second composition for at least 15 minutes, at a temperature of from about 25° C. to about 100° C., and optionally combining any processing aids to said second composition;
j) optionally combining any scavenger material, structurant, and/or anti-agglomeration agent with said second composition during step or thereafter; and
k) optionally spray drying said or agglomeration of the second composition.
